(19) United States
(12) Patent Application Publication
(10) Pub. No.: US 2021/0163308 A1
(43) Pub. Date: Jun. 3, 2021

(54) COMPOSITIONS AND METHODS FOR THE MODULATION OF CYTOKINES

(71) Applicant: GALLEON LABS LLC, Naples, FL (US)

(72) Inventors: Siva Gudi, San Diego, CA (US); Suzana Corritori, San Clemente, CA (US)

(21) Appl. No.: 17/176,728

(22) Filed: Feb. 16, 2021

Related U.S. Application Data
(63) Continuation of application No. PCT/US2019/046635, filed on Aug. 15, 2019.
(60) Provisional application No. 62/719,334, filed on Aug. 17, 2018.

(51) Int. Cl. C01F 11/02; C07C 49/747; C07C 59/10; C07C 229/06

(52) U.S. Cl. CPC C01F 11/02; C07C 229/06; C07C 59/10; C07C 49/747

(57) ABSTRACT

A method for modulating production of a cytokine, or molecule upregulated or down regulated by a cytokine, in a patient in need thereof, including administering to a patient a composition comprising at least one of a strontium-containing compound, a cysteine-based antioxidant, a polyhydroxyphenol and beta hydroxybutyric acid to the patient, whereby an amount of a cytokine in the patient is modulated.

### CROSS-REFERENCE TO RELATED APPLICATIONS

This application is a continuation of International Application No. PCT/US2019/046635, filed Aug. 15, 2019, and published on Feb. 20, 2020 as WO 2020/037114, which claims the benefit of U.S. Provisional Application No. 62/719,334, filed on Aug. 17, 2018, the disclosures of which are incorporated herein by reference in their entireties

### TECHNICAL FIELD

The present disclosure relates to therapeutically-active compositions and formulations of strontium for modulating (increasing or decreasing) the production of cytokines and other biologically active molecules involved in inflammation, immunomodulation, and tissue remodeling process.

### BACKGROUND

Cytokines are small molecules that are secreted by cells specifically affecting the interactions and communications between cells and include interleukins, interferons, colony-stimulating factors, and growth factors. Cytokines are produced by many different cell types and often show overlapping activities regulating proliferation or differentiation, depending on the type and developmental state of the target cells involved. Cytokines are shown to be involved in many important biological responses in the body in health and disease, being secreted in response to numerous stimuli including inflammation, modification or regulation of immune function (immunomodulation) and tissue remodeling that occur in wound healing as a response to tissue injury.

With regard to inflammation, there are both pro-inflammatory cytokines and anti-inflammatory cytokines Inflammation is characterized by an interplay between pro- and anti-inflammatory cytokines. Pro-inflammatory cytokines are produced predominantly by activated macrophages and are involved in the up-regulation of inflammatory reactions. Examples of pro-inflammatory cytokines include but are not limited to interleukin-1 (IL-1), interleukin-6 (IL-6), interleukin-17 (IL-17), tumor necrosis factor-α (TNF-α). The cytokines interleukin-6 (IL-6), IL-1, IL-17, and TNFα are elevated in most, if not all, inflammatory conditions, and have been recognized as targets of therapeutic intervention. Cytokines that are generally recognized as anti-inflammatory include but are not limited to Interleukin-4 (IL-4), Interleukin-10 (IL-10), Interferon-alpha (INF-alpha), and transforming growth factor-beta (TGF-beta) Inflammation is recognized as one of the common denominators of numerous diseases. Every chronic disease is basically an inflammatory disease.

Inflammatory responses in the peripheral and central nervous systems play key roles in the development and persistence of many pathological painful conditions. There is significant evidence showing that certain cytokines/chemokines are involved in not only the initiation but also the persistence of pathologic pain by directly activating nociceptive sensory neurons. Certain inflammatory cytokines are also involved in nerve-injury/inflammation-induced central sensitization, and are related to the development of contralateral hyperalgesia/allodynia.

A hallmark of immunity is the production of a multi-faceted array of inflammatory cytokines that influence the innate and adaptive immune responses. Cytokines play a key role in modulation of immune responses. Cytokines that play a major role in the innate immunity include TNF-alpha, Il-1, IL-10, IL-12, type I Interferons (INF-alpha and -beta), and chemokines. Other important molecules involved in immunomodulation include human leukocyte antigen-antigen D related (HLA-DR), soluble immunoglobulin G (sIgG), soluble interleukin 17A (sIL-17A), soluble interleukin 17F (sIL-17F), soluble interleukin 6 (sIL-6), macrophage colony-stimulating factor (M-CSF), soluble interleukin 2 (sIL-2), soluble interleukin 10 (sIL-10), cluster of differentiation 40 (CD40) and cluster of differentiation 69 (CD69). Many pathological conditions are characterized by an abnormal immune response with substantial involvement of numerous cytokines. Major pathological conditions that have immune component with immunomodulatory cytokines involvement include but are not limited to rheumatoid arthritis, asthma, inflammatory bowel disease, psoriasis.

Wound healing and repair is a complex and dynamic process of replacing devitalized and missing cellular structures and tissue layers. It includes a complex and coordinated series of events that include chemotaxis, phagocytosis, neocollagenesis, collagen degradation, and collagen remodeling, supported further by angiogenesis, epithelization, and the production of new glycosaminoglycans and proteoglycans. Tissue remodeling is considered as the final process of the wound healing/repair process during which replacement of skin structures with fibroblastic mediated scar tissue takes place. Cytokines and other molecules involved in tissue remodeling include TGF-beta, Platelet-derived growth factor (PDGF), IL-4, IL-13, 11-10, IL-17, monocyte chemotactic protein-1 (MCP-1)urokinase-type plasminogen activator (uPAR or CD87), matrix metalloproteinase 1 (MMP-1), matrix metalloproteinase 9 (MMP-9), plasminogen activator inhibitor 1 (PAI-I), tissue plasminogen activator (tPA), matrix metalloproteinase 3 (MMP-3), Keratin 8/18, TIMP metallopeptidase inhibitor 2 (TIMP-2), basic fibroblast growth factor (bFGF), Collagen-III, alpha smooth muscle actin antibody (α-SMA), Collagen-IV, epidermal growth factor receptor (EGFR), Collagen-I and TIMP metallopeptidase inhibitor 1 (TIMP-1).

### SUMMARY

In view of the diverse processes in which cytokines and related molecules are involved, there is always a need for compounds and compositions capable of modulating their effects. Such compositions are provided herein.

The following simplified summary provides a basic understanding of some aspects of the claimed subject matter. This summary is not an extensive overview, and is not intended to identify key/critical elements or to delineate the scope of the claimed subject matter.

One embodiment is a method for modulating production of a cytokine, or molecule upregulated or down regulated by a cytokine, in a patient in need thereof, comprising administering to a patient a composition comprising at least one compound selected from the group consisting of a strontium-containing compound, a cysteine-based antioxidant, a polyhydroxyphenol and beta hydroxybutyric acid to the patient, whereby an amount of a cytokine in said patient is modulated. In one aspect, the cytokine is associated with inflammation. In other embodiments, the cytokine or molecule upregulated or down regulated by said cytokine is Eotaxin-3, soluble tumor necrosis factor-alpha (sTNF-α), vascular cell adhesion molecule 1 (VCAM-1), interleukin-1 alpha (IL-1α), interferon gamma-induced protein 10 (IP-10), interferon-inducible T-cell alpha chemoattractant (I-TAC), interleukin-8 (IL-8), serum amyloid A protein (SAA), monocyte chemoattractant protein-1 (MCP-1), prostaglandin E2 (sPGE2), intercellular adhesion molecule 1 (ICAM-1), monokine induced by interferon-gamma (MIG), E-selectin, P-selectin, macrophage inflammatory protein 1 (MIP-1) or interleukin 6 (IL-6). In one aspect, the cytokine or molecule upregulated or downregulated by the cytokine is associated with acne, psoriasis, rosacea, dermatitis or eczema. In another aspect, the cytokine or molecule upregulated or downregulated by the cytokine is associated with immunomodulation. In another aspect, the cytokine or molecule upregulated or downregulated by the cytokine is human leukocyte antigen-antigen D related (HLA-DR), soluble immunoglobulin G (sIgG), soluble interleukin 17A (sIL-17A),), soluble interleukin 17F (sIL-17F),), soluble interleukin 6 (sIL-6), macrophage colony-stimulating factor (M-CSF), soluble interleukin 2 (sIL-2), soluble interleukin 10 (sIL-10), cluster of differentiation 40 (CD40) or cluster of differentiation 69 (CD69). In another embodiment, the cytokine or molecule upregulated or down regulated by the cytokine is associated with tissue remodeling. In another embodiment, the cytokine or molecule upregulated or down regulated by the cytokine is urokinase-type plasminogen activator (uPAR or CD87), matrix metalloproteinase 1 (MMP-1), matrix metalloproteinase 9 (MMP-9), plasminogen activator inhibitor 1 (PAI-I), tissue plasminogen activator (tPA), matrix metalloproteinase 3 (MMP-3), Keratin 8/18, TIMP metallopeptidase inhibitor 2 (TIMP-2), basic fibroblast growth factor (bFGF), Collagen-III, alpha smooth muscle actin antibody (α-SMA), Collagen-IV, epidermal growth factor receptor (EGFR), Collagen-I or TIMP metallopeptidase inhibitor 1 (TIMP-1). In another embodiment, the tissue remodeling is associated with tissue injury or trauma. In one aspect, the strontium-containing compound is strontium nitrate, strontium chloride or strontium chloride hexahydrate. In another aspect, the polyhydroxyphenol is gallic acid, caffeic acid, tannic acid, epicatechin, epigallocatechin gallate, epigallocatechin, epicatechin gallate, ellagic acid, myricetin, luteolin, naringen, genistein, apagenin, nordihydroguaiaretic acid, or esters thereof. In another embodiment, production of the cytokine or the molecule upregulated or downregulated by the cytokine is increased. In another aspect, production of the cytokine or the molecule upregulated or downregulated by the cytokine is decreased. In one embodiment, the composition is topically administered. In another embodiment, the composition is orally administered. In one aspect, the composition is in the form of a tablet, pill, dragee, capsule, liquid, gel, syrup, elixir, slurry or suspension. In one aspect, the composition consists of a strontium-containing compound, a cysteine-based antioxidant and a polyhydroxyphenol. In another aspect, the strontium-containing compound is strontium chloride, the cysteine-based antioxidant is N-acetyl cysteine, and the polyhydroxyphenol is gallic acid. In one embodiment, the composition consists of a strontium-containing compound, a cysteine-based antioxidant and beta hydroxybutyrate. In another embodiment, the strontium-containing compound is strontium hydroxide and the cysteine-based antioxidant is N-acetyl cysteine.

### BRIEF DESCRIPTION OF THE DRAWINGS

FIG. 1 is a chart illustrating cell types, disease/tissue relevance and biomarker endpoints in the screening in the first two studies described herein.

FIG. 2A is a graph that provides an explanation of a screened compound profile is the assay system described herein.

FIG. 2B is a graph providing further explanation of the assay system described herein.

FIG. 2C is a graph providing further explanation of the assay system described herein

FIG. 3 provides explanation of a profile data table.

FIG. 4 shows the profile of strontium nitrate in a screening assay at eight concentrations.

FIG. 5 shows the top database search result for strontium nitrate.

FIG. 6 shows the second database search result for strontium nitrate.

FIGS. 7-8 shows the top reference database matches for strontium nitrate.

FIG. 9A shows the screening profile of gallic acid.

FIG. 9B shows the screening profile of gallic acid at non-cytotoxic concentrations.

FIG. 10 shows the top database search result for gallic acid.

FIG. 11 shows an additional database search result for gallic acid.

FIGS. 12-13 show the top reference database matches for gallic acid.

FIG. 14 shows the screening profile of beta hydroxybutyric acid.

FIG. 15 shows the top database search result for beta hydroxybutyric acid.

FIG. 16 shows a second database search result for beta hydroxybutyric acid.

FIGS. 17-18 show the top reference database matches for beta hydroxybutyric acid.

FIG. 19 shows the screening profile of N-acetyl cysteine.

FIG. 20 shows the top database search result for N-acetyl cysteine.

FIG. 21 shows a second database search result for N-acetyl cysteine.

FIG. 22 shows the top reference database matches for N-acetyl cysteine.

FIG. 23 shows a clustering of project profiles.

FIG. 24 shows the screening profile of strontium nitrate.

FIG. 25 shows the top database search result for strontium nitrate.

FIG. 26 shows a second database search result for strontium nitrate.

FIG. 27 shows the top reference database matches for strontium nitrate.

FIG. 28 shows the screening profile of gallic acid.

FIG. 29 shows the top database search result for gallic acid.

FIG. 30 shows a second database search for gallic acid.

FIG. 31 shows the top reference database matches for gallic acid.

FIG. 32A shows a first screening profile of beta hydroxybutyric acid.

FIG. 32B shows a second screening profile of beta hydroxybutyric acid.

FIG. 33 shows the top database search result for beta hydroxybutyric acid.

FIG. 34 shows a second database search result for beta hydroxybutyric acid.

FIG. 35 shows the top reference database matches for beta hydroxybutyric acid.

FIG. 36A shows a first screening profile of N-acetyl cysteine.

FIG. 36B shows a second screening profile of N-acetyl cysteine.

FIG. 37 shows the top database search result for N-acetyl cysteine.

FIG. 38 shows a second database search result for N-acetyl cysteine.

FIG. 39 shows the top reference database matches for N-acetyl cysteine.

FIG. 40 shows the clustering of project profiles.

FIG. 41 is a chart illustrating cell types, disease/tissue relevance and biomarker endpoints in the screening in the third study described herein.

FIG. 42 shows the screening profile of strontium nitrate, anhydrous.

FIG. 43 shows the screening profile of strontium nitrate hexahydrate.

FIG. 44A shows a first screening profile of Formula 787.

FIG. 44B shows a second screening profile of Formula 787.

FIG. 45 shows the screening profile of Formula F11b.

FIG. 46 shows a first screening profile of aspartame.

FIG. 47 shows a second screening profile of aspartame.

FIG. 48 shows a top database search result for aspartame (FSL-1).

FIG. 49 shows a second database search result for aspartame (LPS).

FIG. 50 shows a top database search result for aspartame (resquimod, 100 μM).

FIG. 51 shows a top database search result for aspartame (resquimod, 33 μm).

FIG. 52 shows a first set of top reference database matches for aspartame.

FIG. 53 shows a second set of top reference database matches for aspartame.

FIG. 54 shows a summary of a first set of mechanisms of action/therapeutic uses for various compounds.

FIG. 55 shows a summary of a second set of mechanisms of action/therapeutic uses for various compounds.

### DETAILED DESCRIPTION OF THE PREFERRED EMBODIMENT

The present disclosure relates to therapeutically-active compounds, compositions and methods for modulating the production of cytokines and other molecules involved in inflammation, tissue remodeling and immunomodulatory activities.

The present disclosure also relates to therapeutically-active compositions that combine strontium with at least one additional compound that synergistically increases the overall therapeutic potency of the combination beyond the potency of any of the separate constituents.

The term “modulating” means that the compounds and compositions either increase or decrease production or release of a particular cytokine. The term “other molecules” encompasses molecules that do not fall within the definition of a “cytokine”, but are upregulated or downregulated by one or more cytokines. Such molecules include cell adhesion molecules (ICAM-1, VCAM-1, E-selectin, P-selectin), protein kinases (e.g., MAP kinases), matrix metallopeptidases (MMPs), plasminogen activators (tPA and uPA), and the like.

Cytokine signal strength alteration may occur both as an increase and a decrease with varying concentrations of compound. The therapeutically-active compositions include one or more of a strontium-containing compound, a cysteine-based antioxidant, a polyhydroxyphenol and beta hydroxybutyric acid (BHB). The compositions may be administered in vivo, or to cells in vitro or ex vivo.

### Definitions

In the description that follows, a number of terms are extensively utilized. The following non-limiting definitions provide a clear and consistent understanding of the specification and claims, including the exemplary scope to be given such terms. Unless defined otherwise, all technical and scientific terms used herein have the same meaning as is commonly understood by one of ordinary skill in the art to which this invention belongs.

The terms “invention” or “present invention” as used herein are intended to be non-limiting and are not intended to refer to any single embodiment of the particular invention but encompasses all possible embodiments as described in the specification and the claims.

As used herein, the term “and/or” may mean “and,” it may mean “or,” it may mean “exclusive-or,” it may mean “one,” it may mean “some, but not all,” it may mean “neither,” and/or it may mean “both.”

The term “epithelial” or “epithelium” as used herein refers to outer surfaces of the body in the broadest sense of the word and therefore implicitly includes all keratinized tissue as well as mucous membranes, for example, the mouth, throat, surfaces of the eye, the respiratory tract, the gastrointestinal tract, and the genitourinary tract, including the cervix and the vagina.

The term “beneficial agent” as used herein refers to a chemical, compound, or ingredient that helps reduce pain, pruritus, or inflammation and/or promotes healing in epithelial tissue and/or improves fibrotic conditions. Beneficial agents may be chemicals or compounds that are either generally recognized as safe, approved by the U.S. Food and Drug Administration (or equivalent agency in other countries), or recognized by those skilled in the arts as being beneficial. Non-limiting examples of beneficial agents are described and listed herein, including analgesics, antihistamines, antibacterials, corticosteroids, moisturizers, vitamins, biologics, plant extracts, and polymers.

As used herein, “treatment” means any manner in which the symptoms of a condition, disorder or disease are ameliorated or otherwise beneficially altered. Treatment also encompasses any pharmaceutical use of the compositions herein.

As used herein, “subject” refers to an animal, including, but not limited to, a primate (e.g., human). The terms “subject” and “patient” are used interchangeably herein.

As used herein, the term “strontium-containing component” refers to either elemental strontium or a strontium salt. The terms “elemental strontium” and “strontium cation” are used interchangeably herein.

As used herein, the terms “moisturizer” and “skin protectant” are used interchangeably unless otherwise indicated.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. For example all tautomers of phosphate groups are intended to be included. Furthermore, all tautomers of heterocyclic bases known in the art are intended to be included, including tautomers of natural and non-natural purine-bases and pyrimidine-bases.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

Where a range of values is provided, it is understood that the upper and lower limit, and each intervening value between the upper and lower limit of the range is encompassed within the embodiments.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element in a compound may be any isotope of said element. Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

The term “salt” as used herein is a broad term and includes without limitation pharmaceutically acceptable salts such as a salt of a compound that does not cause significant harm to an organism to which it is administered and does not abrogate the biological activity and properties of the compound. In some embodiments, the salt is an acid addition salt of the compound. Pharmaceutical salts can be obtained by reacting a compound with an inorganic acid, an organic acid, or a base. Suitable pharmaceutically acceptable salts include metallic salts, organic salts, salts of free acids and bases, inorganic salts, and other salts which are currently in widespread pharmaceutical use and are listed in sources well known to those of skill in the art, such as, for example, The Merck Index.

The present disclosure relates to therapeutically-active compositions and methods for modulating (increasing or decreasing) the production of cytokines and other molecules involved in inflammation, tissue remodeling and immunomodulatory activities. The therapeutically-active compositions include one or more of a strontium-containing compound, a cysteine-based antioxidant, a polyhydroxyphenol and beta hydroxybutyric acid (BHB). The compositions are also capable of inhibiting proliferation of various cell types, including coronary artery smooth muscle cells, endothelial cells, T cells and fibroblasts. As illustrated in the data presented herein below, these compounds are capable of modulating cytokines, including those disclosed hereinabove. The ability of the compounds disclosed herein to modulate the production of other cytokines involved in inflammation, immunomodulation and tissue remodeling is also within the scope of the present disclosure, and the ability of these compounds to modulate other cytokines can easily be determined by one of ordinary skill in the art using the screening methods disclosed herein. The compositions and methods disclosed herein are capable of treating any disorder involving the increase or decrease in production of one or more cytokines if the increase or decrease is associated with the disorder, and if the compositions and methods are capable of preventing the increase or decrease. For example, TNF-α is often upregulated in inflammatory conditions, so the ability of one or more of the compounds and compositions disclosed herein to decrease TNF-α production indicates that the compound would be effective in treating the particular inflammatory condition in which TNF-α is upregulated.

Abbreviations: GPR: g-protein coupled receptor; GSH: reduced glutathione; GSSG: oxidized glutathione; HDAC: histone deacetylase; IL: interleukin; MAPK: mitogen activated protein kinase; MCP: monocyte chemotactic protein; MMP: matrix metalloproteinase; NFkB: nuclear factor kappa light chain enhancer of activated B cells; NLRP: NOD-like receptor family, pyrin domain containing; SNAP: synaptosomal associated protein; SNARE: soluble NSF attachment protein receptor; SRC: sarcoma; TLR: toll-like receptor; TNF: tumor necrosis factor; TRPA: transient receptor potential cation channel; TRPV: transient receptor potential cation channel subfamily V; VGLUT: vesicular glutamate transporter; VNUT: vesicular nucleotide transporter.

The following technical publications include disclosures relating to cytokines or cytokine modulation. Each reference is hereby incorporated in its entirety: “Activation and regulation of the inflammasomes,” Latz, et al., Nat Rev Immunol., 2013 vol. 13, no. 6, p. 1-31; “All-Trans-Retinoic Acid Induces Interleukin-8 via the Nuclear Factor-jB and p38 Mitogen-Activated Protein Kinase Pathways in Normal Human Keratinocytes,” Dai, et al., J Invest Dermatol., 2004, vol. 123, p. 1078-85; “Anticonvulsant Effects of β-Hydroxybutyrate in Mice,” Yum, et al., J of Epilepsy Res. 2012, vol. 2, no. 2, p. 29-32; “Ketone body β-hydroxybutyrate blocks the NLRP3 inflammasome-mediated inflammatory disease,” Youm, et al., 2015 vol. 21, no. 3, p. 23-9; “Cellular Metabolism Modulates Ion Channels That Regulate Neuronal Excitability,” Lutas, dissertation Harvard University, Division of Medical Sciences, 2015; “Calcium-Permeable Ion Channels In Pain Signaling,” Bourinet, Physiol Rev. 2014, vol. 94, p. 81-140; “Cell cycle progression is regulated by intertwined redox oscillators,” da Veiga Moreira, et al., Theoretical Biology and Medical Modeling, 2015, vol. 12, no. 10, p. 1-14; “Changes in TRP Channels Expression in Painful Conditions,” Bishnoi and Premkumar, The Open Pain J. 2013, vol. 6, suppl. 1:M3, p. 10-22; “Clinical utility of N-acetylcysteine in chronic obstructive pulmonary disease,” Tse, et al., International J COPD., 2014, vol. v9, p. 825-36; “D-beta-hydroxybutyrate extends lifespan in C. elegans,” Edwards, et al., Aging, 2014, vol. 6, no. 8, p. 621-44; “Effect of Strontium Chloride on Experimental Bladder Inflammation in Rat,” Korgali, et al., International Scholarly Research Notices, 2014, p. 1-6; “Gallic Acid Inhibits Histamine Release and Pro-inflammatory Cytokine Production in Mast Cells,” Kim, et al., Toxicological Sciences, 2006, vol 91, no. 1, p. 123-31; “Hydroxycarboxylic acid receptor 2 mediates dimethyl fumarate's protective effect in EAE,” Chen, et al., J Clin Investigation., 2014, vol. 124, no. 5, p. 2188-92; “In pursuit of small molecule chemistry for calcium-permeable non-selective TRPC channel,” Bon and Beech, British J Pharmacology. 2013, vol. 170, p. 459-74; “Influence Of N-Acetylcysteine On Icam-1 Expression And Il-8 Release From Endothelial And Epithelial Cells,” Radomska-Lesniewska, et al., J Physiology Pharmacol. 2006, vol. 57, supp. 4, p. 325-34; “Inhibitors of histone deacetylase arrest cell cycle and induce apoptosis in cervical carcinoma cells circumventing human papillomavirus oncogene expression,” Finzer, et al., Oncogene, 2001, vol. 20, p. 4768-76; “Interleukin-1β in Central Nervous System Injury and Repair,” Hewett, et al., Eur J Neurodegener Dis. 2012, vol. 1, no. 2, p. 195-211; “International union of basic and clinical pharmacology. XCVI. Pattern recognition receptors in health and disease,” Bryant, et al., Pharmacological Rev. 2015, vol. 67, p 462-5004; “Ion conduction and selectivity in acid-sensing ion channel 1,” Yang and Palmer, J Gen Physiol. 2014, vol. 144, no. 3, p. 245-55; “The ketogenic diet as a treatment paradigm for diverse neurological disorders,” Stafstrom and Rho, Frontiers in Pharmacology, 2012, vol. 3, article 59, p. 1-8; “Mechanism-based treatment for chemotherapy-induced peripheral neuropathic pain,” Sisignano, et al., Nat Rev Neurol, online publication 4 Nov. 2014. “Integration of the metabolic/redox state, histone gene switching, DNA replication and S-phase progression by moonlighting metabolic enzymes,” He, et al., Biosci Rep. 2013, vol. 33, p. 187-97.; “Neutrophil apoptosis mediated by nicotinic acid receptors (GPR109A),” Kostylina, et al., Cell Death and Differentiation, 2008, vol. 15, p. 134-42; “NLRP3 inflammasome and its inhibitors: a review,” Shao, et al., Frontiers in Pharmacology, 2015, vol. 6, article 262, p. 1-9; “Retinoic Acid Inhibits Induction of c-Jun Protein by Ultraviolet Radiation that Occurs Subsequent to Activation of Mitogen-activated Protein Kinase Pathways in Human Skin In Vivo,” Fisher, et al., J Clin Invest., 1998, vol. 101, no. 6, p. 1432-40; “Retinoids activate the irritant receptor TRPV1 and produce sensory hypersensitivity,” Yin, et al., J Clin Invest. 2013, vol. 123, no. 9, p. 3941-51; “Role of Reactive Oxygen Species and Redox in Regulating the Function of Transient Receptor Potential Channels,” Song, et al., Antioxidants & Redox Signaling, 2011, vol. 15, no. 6, p. 1549-66; “Role of β-hydroxybutyrate, its polymer poly-β-hydroxybutyrate and inorganic polyphosphate in mammalian health and disease,” Dedkova and Blatter, Frontiers in Physiology, 2014, vol. 5, article 260, p. 1-22; “ROS-Mediated NLRP3 Inflammasome Activity Is Essential for Burn-Induced Acute Lung Injury,” Han, et al., Mediators of Inflammation, 2015, article 720457, p. 1-16; “Strontium Chloride: Can It Be a New Treatment Option for Ulcerative Colitis?,” Topal, et al., BioMed Research International, 2014, article 530687, p. 1-5; “The ketogenic diet increases mitochondrial glutathione levels,” Jarrett, et al., J Neurochemistry., 2008, vol. 106, p. 1044-51; “The role of HCA2 (GPR109A) in regulating macrophage function,” Zandi-Nejad, et al., FASEB J., 2013, vol. 27, p. 4366-74; The Therapeutic Potential of Modifying Inflammasomes and NOD-Like Receptors,” Virgilio, Pharmacological Rev., vol. 65, p. 872-905; “TLR2 Expression Is Increased in Rosacea and Stimulates Enhanced Serine Protease Production by Keratinocytes,” Yamasaki, et al., 2011, vol. 131, no. 3, p. 68-97; “TRP channels in the skin,” Toth, et al., British J Pharmacol., 2014, vol. 171, p. 2568-81; “TRP-channels as key integrators of lipid pathways in nociceptive neurons,” Sisignano, et al., Progress in Lipid Research, 2014, vol. 53, p. 93-107.

### Strontium

Strontium is present as a divalent cation. Strontium is designated by its commonly used atomic symbol, ‘Sr.’ Strontium is available as an inorganic or organic salt which is water soluble at room temperature in the range of 1 to 100 g/l. Inorganic salts include, for example, strontium chloride, strontium sulfate, strontium carbonate, strontium nitrate, strontium hydroxide, strontium hydrosulfide, strontium oxide, strontium acetate, etc. Organic salts include, for example, negatively charged organic acid such as a mono-, di-, tri- or quatro-carboxylic acid, or an amino carboxylic acid that may have a linear or branched carbon chain of from 2 to 30 carbon atoms and one or more amino groups attached thereto. The amino carboxylic acid may be a natural or synthetic amino acid. Examples of organic strontium salts include, for example strontium glutamate, strontium aspartate, strontium malonate, strontium maleate, strontium citrate, strontium threonate, strontium lactate, strontium pyruvate, strontium ascorbate, strontium alpha-ketoglutarate or strontium succinate. Other examples of strontium salts, and methods for preparation thereof, can be found, for example, in US Patent Application Pub. No. 2010/0048697.

### Cysteine-Based Antioxidants

The term “cysteine-based” includes cysteine and cystine. Alternatively the cysteine-based compound is acetylated at the amino group of the cysteine to produce N-acetyl-cysteine, commonly abbreviated acetylcysteine or NAC. Non-limiting examples of cysteine based antioxidants include: cysteine, cystine, acetylcysteine, diacetylcysteine, and esters thereof.

Cysteine exists in two enantiomeric forms, designated ‘L-cysteine’ and ‘D-cysteine’, of which the L form is used in living organisms while the D form is not. While both the L and D forms are contemplated in the present disclosure, the L form of acetylcysteine is most preferred, i.e., NAC. If the D form of NAC is intended, it will be referred to as D-NAC. In addition, both L-Cys and D-Cys can form disulfide bonds between the two thiol groups to form a ‘dimer’, literally a pair of Cys molecules. Such disulfide bonds occur in many proteins and play a critical regulatory role in biochemical pathways due to the ease of their reversible formation by oxidative processes and dissolution by reductive processes. By convention, a disulfide-bonded dimer of cysteine is termed cystine. Thus one cysteine molecule under appropriate reducing conditions or enzymatic processing will yield two cysteine molecules. Cystine can be formed from either two L-Cys molecules, two D-Cys molecules, or one L-Cys and one D-Cys molecules. Another exemplary cysteine-based compound is N,S-diacetylcysteine. All of such variants are incorporated within the present disclosure.

### Polyhydroxyphenols

Polyhydroxyphenols are phenolic compounds possessing at least two hydroxyl groups, preferably in the ortho and para positions. One exemplary compound is 3,4,5-trihydroxy benzoic acid, also called gallic acid. The term “polyhydroxyphenol” does not include carboxylic acids, such as ranelate. Non-limiting examples of polyhydroxyphenols include: gallic acid, caffeic acid, tannic acid, epicatechin, epigallocatechin gallate, epigallocatechin, epicatechin gallate, ellagic acid, myricetin, luteolin, naringen, genistein, apagenin, nordihydroguaiaretic acid, and esters thereof.

The polyhydroxyphenol can be added to the compositions described herein in essentially purified form, or they can be added in the form of polyhydroxyphenol-containing plant extracts, such as green tea and soy extracts.

The flavonoids are polyphenolic compounds possessing 15 carbon atoms; two six-carbon benzyl rings that are usually joined together by a linear, saturated three carbon chain. Other flavonoids may consist of two benzyl rings joined together by a third 5- or 6-carbon ring structure. Flavonoids constitute one of the most characteristic classes of compounds in higher plants. Many flavonoids are easily recognized as the pigments in flowering plants.

The monomeric phenolic compounds, e.g. gallic acid and caffeic acid, have a carboxylic acid group, which may be esterified with a sugar moiety such as glucose. In the case of gallic acid, such esterification produces glucogallin. Other organic esters may also be effective, such as the ethyl ester of gallic acid, ethyl gallate, or the propyl ester of gallic acid, propyl gallate.

Also contemplated by the present disclosure are polymeric phenolic compounds that have two or more aromatic rings that typically, but do not necessarily have the same structure. One such example is reservatrol. Another is pentagalloyl glucose, which consists of five gallic acid residues that are esterified to one glucose molecule. This molecule will be cleaved in vivo by non-specific esterases, which free the individual gallic acid residues. The use of such forms of polyhydroxyphenolic compounds has the added advantage of lowering osmotic activity, since one molecule of pentagalloyl glucose produces one unit of osmotic activity, as compared to five units of osmotic activity produced by the use of five separate molecules of gallic acid.

Tannic acid is another example of a high molecular weight gallic acid polymer in which one or more esterified gallic acid residues are esterified to a central glucose molecule.

Ellagic acid is an example of a gallic acid dimmer. While this molecule no longer possesses the gallic acid-like phenolic structure, it does maintain many of the same bioactivities of gallic acid and is thus useful in the practice of the present disclosure.

Compounds having a flavone backbone include, for example, quercetin, and epicatechin (EC) and derivatives thereof, such as epigallocatechin gallate (EGCG found in green tea), epigallocatechin (EGC) and epicatechin gallate (ECG).

Other polyhydroxyphenolic compounds include, for example, myricetin, luteolin, naringen, genistein and nordihydroguaiaretic acid (NDGA).

In one particular embodiment, the polyhydroxyphenols that are useful also exhibit one or more carboxyl groups, such as gallic acid. The carboxyl group can serve as an additional counterion, and also assist in matrix formation with an optional polyanionic polymer.

Also contemplated is a composition that incorporates a combination of strontium with a mixture of polyhydroxyphenols, for example one or more, or two or more, of the polyhydroxyphenols noted or discussed herein and above. Using more than one polyhydroxyphenol has a synergistic effect due to the differential activities of each polyhydroxyphenol. It is contemplated that this synergistic effect has enhanced efficacy in treating sensory irritation that includes but is not limited to pain, pruritus, and development of neuropathic diseases. In one embodiment, the mixture includes monophenolic and polyphenolic polyhydroxyphenols. In one embodiment, the mixture includes monophenolic and biphenolic polyhydroxyphenols. In another embodiment, the mixture includes monophenolic and triphenolic polyhydroxyphenols. In another embodiment, the mixture includes biphenolic and triphenolic polyhydroxyphenols. In another embodiment, the mixture includes monophenolic, biphenolic, and triphenolic polyhydroxyphenols. In another embodiment, the mixture includes an ATP analogue with a monophenolic, biphenolic, or triphenolic polyhydroxyphenol. In yet another embodiment the mixture of polyhydroxyphenols is gallic acid and caffeic acid. In another embodiment, the mixture of polyhydroxyphenols is myricetin and caffeic acid. In another embodiment, the mixture of polyhydroxyphenols is myricetin and gallic acid. In another embodiment, the mixture of polyhydroxyphenols is myricetin, gallic acid, and caffeic acid.

### Beta Hydroxybutyrate

Beta hydroxybutyrate, (also known as beta hydroxybutyric acid, beta hydroxybutanoic acid, 3-hydroxybutyrate, 3-hydroxybutyric acid, and 3-hydroxybutanoic acid and collectively referred to as “BHB”) (chemical formula CH3CH(OH)CH2CO2H; CAS no. 300-85-6) is a beta hydroxy acid. BHB has several isoforms all of which are contemplated, including D, L, R (racemic mixture of D and L), and natural forms. BHB can be a salt form or polymer form. Its biosynthesis is catalyzed by the enzyme beta-hydroxybutyrate dehydrogenase. It is synthesized by the liver during fasting states in humans and is often a used as an indicator for diabetic ketoacidosis. The concentration of beta-hydroxybutyrate, like that of other ketone bodies, is raised in ketosis. The compound can be used as an energy source by the brain when blood glucose is low.

In one embodiment, the compounds described herein are formulated as pharmaceutical compositions. The compounds and compositions disclosed herein may be administered in vitro, ex vivo or in vivo.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990. The pharmaceutical compositions may also be administered to other mammals, including dogs, cats, sheep, pigs, horses, cows, and the like. Thus, the veterinary use of these pharmaceutical compositions is also within the scope of the present invention.

Suitable routes of administration may, for example, include oral, rectal, topical, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions described herein may be manufactured in a manner that is itself known, e.g., by means of conventional mixing, dissolving, granulating, dragee-making, levigating, emulsifying, encapsulating, entrapping or tabletting processes.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the compound(s) may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

For topical administration, the compounds may be formulated for administration to the epidermis as ointments, gels, creams, pastes, salves, gels, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally, including sublingually, which include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Suitable pharmaceutical compositions include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions described herein can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 6000 mg of each ingredient, preferably between 1 mg and 5000 mg, e.g. 25 to 5000 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2500 mg and the total daily dosage by parenteral administration will typically be in the range 0.1 to 400 mg. Suitably the compounds will be administered for a period of continuous therapy, for example for a week or more, or for months or years.

In one embodiment, the dose of the pharmaceutical composition comprising is from about 10 to about 50 mg per day.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

The compositions described herein may also be used in the preparation of a medicament for treatment of any of the disorders described above.

### Screening of Compound Libraries to Determine Potential Cytokine Modulation Activities of Test Compounds

Compound libraries were screened to determine which cytokines could be modulated by strontium, N-acetyl cysteine, beta hydroxybutyric acid, gallic acid, a combination of strontium chloride, gallic acid and N-acetyl cysteine in equimolar amounts (Formula 787) and a combination of strontium hydroxide, beta hydroxybutyric acid and N-acetyl cysteine in equimolar amounts (Formula F11b). The activity of compound libraries in modulation of a panel of cytokines was compared to these test compounds and combination of compounds.

A human primary cell disease model using 30+ cell types, 50+ systems and 200+ readouts was used in which biomarker responses to drugs are stored in a database of >3,000 drugs. Custom informatics tools were then used to predict mechanism, efficacy and toxicity. Profiling was performed using the following processes:

Annotation: Activities, including relevance to biological pathways and in vivo correlations/predictions are interpreted.

Benchmarking: head-to-head comparison of test compound(s) with a specified reference compound.

Similarity Search: results from unsupervised search for biologically similar compounds from the reference database of >3,000 compounds including biologics, approved drugs and experimental agents.

Cluster Analysis: Cluster analysis interpretation is the graphical display of the similarity of several compounds to one another. Each circle (node) represents the profile of one compound at one dose. Darkest shades represent the highest dose. If nodes are connected, their profiles are correlated (similar). If nodes are unconnected, their profiles are not correlated.

The following primary human cells were used as test systems in Studies CSM001-01-b and CSM002-01-b discussed below.

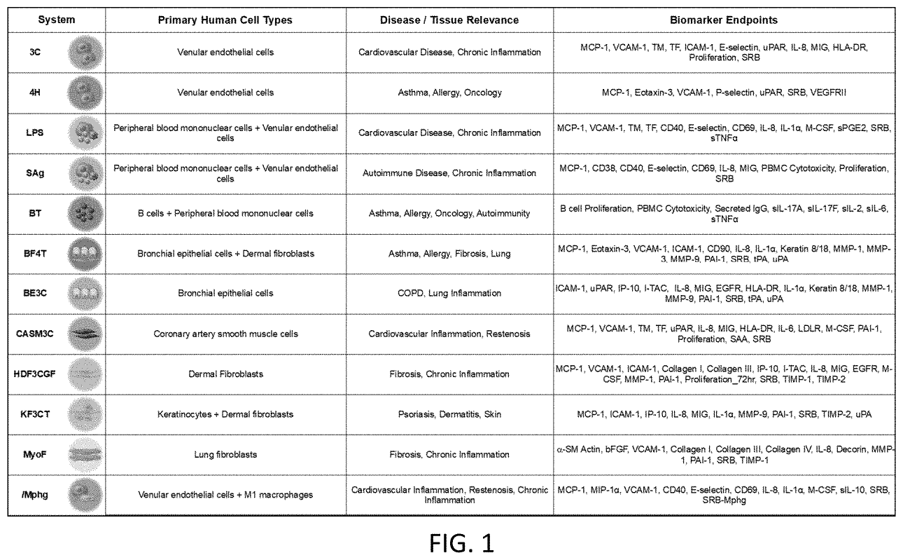

3C (Venular Endothelial cells); 4H (Venular Endothelial Cells); LPS (Peripheral blood mononuclear cells+venular endothelial cells); SAg (Peripheral blood mononuclear cells+venular endothelial cells); BT (B cells+peripheral blood mononuclear cells); BF4T (bronchial epithelial cells+dermal fibroblasts); BE3C (Bronchial epithelial cells); CASM3C (coronary artery smooth muscle cells); HDF3CGF (dermal fibroblasts); KF3CT (keratinocytes+dermal fibroblasts); MyoF (lung fibroblasts); /Mphg (Venular endothelial cells+macrophages). The disease/tissue relevance and biomarker endpoints for these systems is shown in FIG. 1.

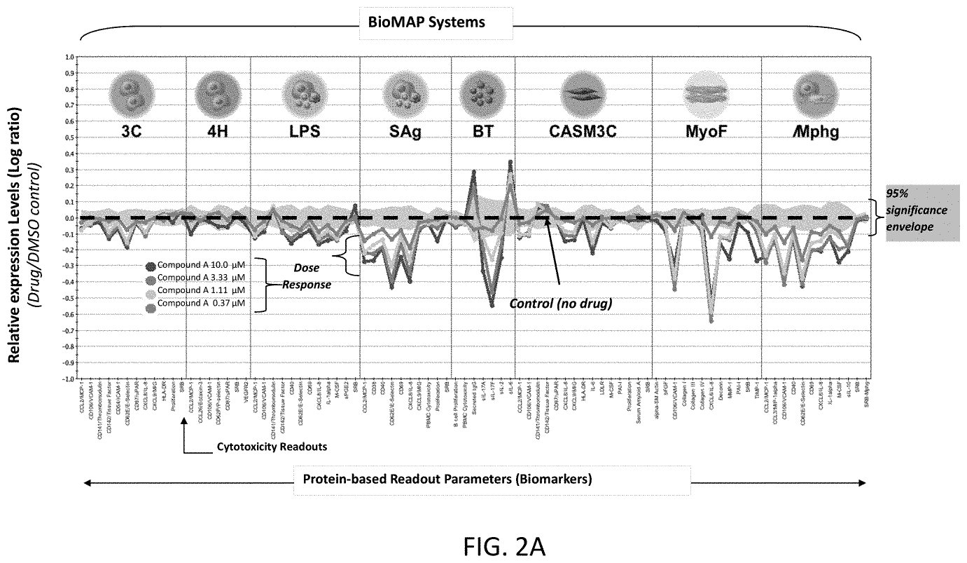

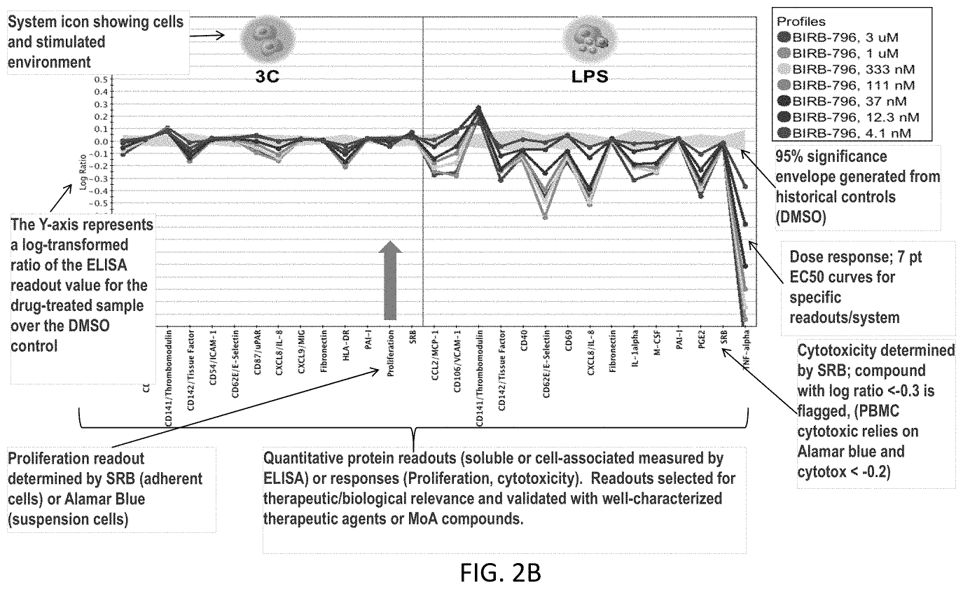

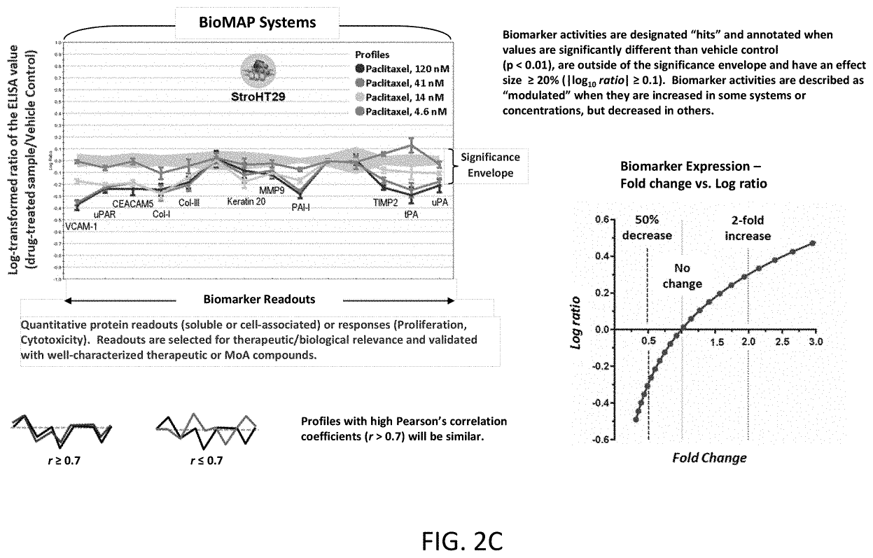

An annotated assay profile was prepared for each test agent. A detailed explanation of an assay profile is shown in FIGS. 2A-2C. Biomarker readouts are annotated when two or more consecutive concentrations are changed in the same direction, relative to the vehicle control, outside of the significance envelope and with an effect size of at least 10% (more than 0.05 log ratio units). Biomarker activities are annotated as “modulated” when they are increased in some system(s) and decreased in other system(s). If applicable, a benchmark overlay was performed at the top non-cytotoxic concentration. Results of a similarity search (all test agents) and clustering (at least three test agents) were determined. Profile data tables were generated in excel file format as a data table of log ratio data (log₁₀ test agent/vehicle control) with a significance envelope at 95% (symmetrical envelope) as mean values and standard deviation. The “hits” table indicates the number of active biomarker readouts. A table of overtly cytotoxic test agents was also prepared which are test agents with a >50% reduction in cellular protein levels (sulforhodamine B, SRB) in one or more systems; and/or >50% reduction in PBMC viability.

A sulforhodamine B (SRB) assay for adherent cells is used to measure cell density based on total cellular protein content of a test well, and is optimized for cytotoxicity of compounds added to cells in a 96-well plate format. Values are linear over a range of cell numbers and a log scale value of −0.3 represents a 50% decrease in total protein levels which is the cytotoxicity threshold value for compounds in the assay systems.

Alamar Blue assays were used to determine the viability of non-adherent PBMCs in the assay systems, and are based on the conversion of resazurin to resorufin via the reduction reactions of metabolically active cells. Resazurin is a nontoxic, cell permeable compound that is blue in color and virtually non-fluorescent. Upon entering cells, resazurin is reduced to resorufin which produces very bright red fluorescence. Viable cells continuously convert resazurin to resorufin, thereby generating a quantitative measure of viability and cytotoxicity. The amount of fluorescence produced is proportional to the number of living cells. Values are linear over a range of cell numbers and a log scale value of less than or equal to −0.1 in SAg or less than or equal to 0.2 in BT represents an approximately 50% decrease in cell viability. This value is the PBMC cytotoxicity threshold for compounds in assay systems.

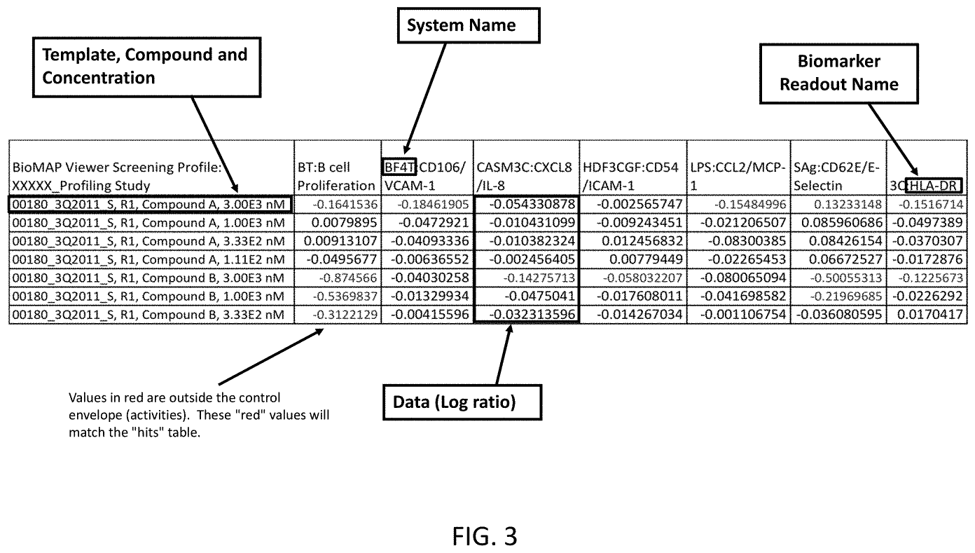

An explanation of a profile data table is illustrated in FIG. 3.

### Study CSM001-01-b

The following compounds were screened in an assay system at the noted concentrations to test for anti-proliferative, inflammation-related, immunomodulatory, tissue remodeling and hemostasis-related activities at the noted concentrations (Table 2). All test compounds were dissolved in dimethyl sulfoxide (DMSO).

TABLE 2

| | Compound | Concentrations tested |
|---|---|---|
| 1 | Strontium Nitrate | 15625 μM, 3125 μM, 625 μM, 125 μM, |
| | | 25 μM, 5 μM, 1 μM, 0.2 μM |
| 2 | Gallic Acid | 1250 μM, 250 μM, 50 μM, 10 μM, 2 μM, |
| | | 0.4 μM, 0.08 μM, 0.016 μM |
| 3 | Beta hydroxybutyric | 1250 μM, 250 μM, 50 μM, 10 μM, 2 μM, |
| | Acid | 0.4 μM, 0.08 μM, 0.016 μM |
| 4 | N-Acetyl - L- Cysteine | 20000 μM, 5000 μM, 1250 μM, 312.5 μM |

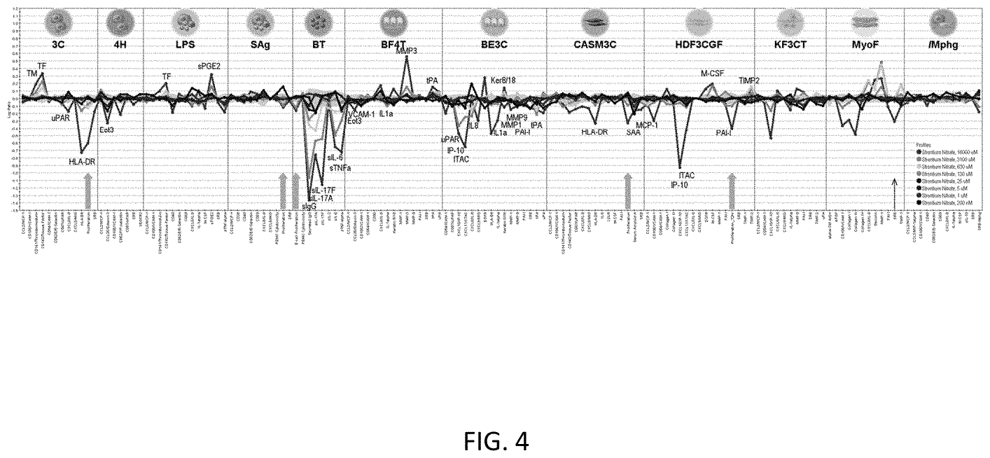

The assay profile of strontium nitrate is shown in FIG. 4. This compound was overtly cytotoxic in MyoF at the top concentration (thin black arrow) and was not annotated. Strontium nitrate was anti-proliferative to endothelial cells, T cells, B cells, coronary artery smooth muscle cells and fibroblasts (grey arrows). This compound also affected inflammation-related activities (decreased Eotaxin-3, sTNFα, VCAM-1, IL-1a, IP-10, I-TAC, IL-8, SAA and MCP-1, and increased sPGE2); affected immunomodulatory activities (decreased HLA-DR, sIgG, sIL-17A, sIL-6, and increased M-CSF); affected tissue remodeling activities (decreased uPAR, MMP-1, MMP-9, PAI-I, tPA; and increased MMP-3, tPA, Keratin 8/18, TIMP-2); and affected hemostatis-related activities (increased TM, TF). Readouts that were significantly modulated in both directions within the same system in a concentration-dependent manner are indicated by thick arrows.

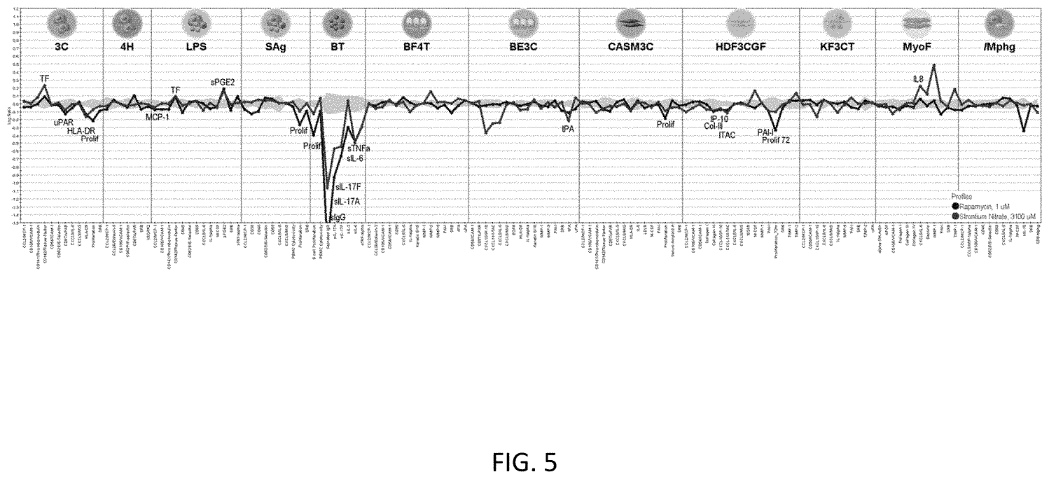

As illustrated in FIG. 5, the top database search result for strontium nitrate was rapamycin which is an inhibitor of mTOR, a serine/threonine protein kinase that regulates cell growth, cell proliferation, cell motility, cell survival, protein synthesis, autophagy, transcription. An overlay of strontium nitrate (3100 μM) and rapamycin (1 μM) had a Pearson correlation r=0.791. Common activities are annotated. The similarity is likely due to the common biology affected.

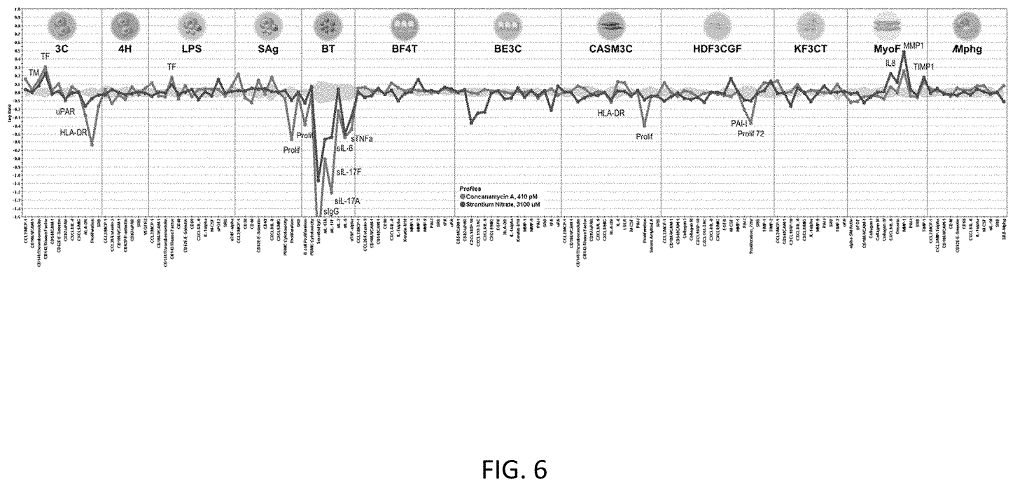

As illustrated in FIG. 6, a second database search result for strontium nitrate was performed. This search identified Concanamycin A which is an antibiotic vacuolar type H⁺-ATPase (V-ATPase) inhibitor. An overlay of strontium nitrate (3100 μM) and Concanamycin A (410 pM) had a Pearson correlation r=0.788. Common activities are annotated. The similarity is likely due to the common biology affected.

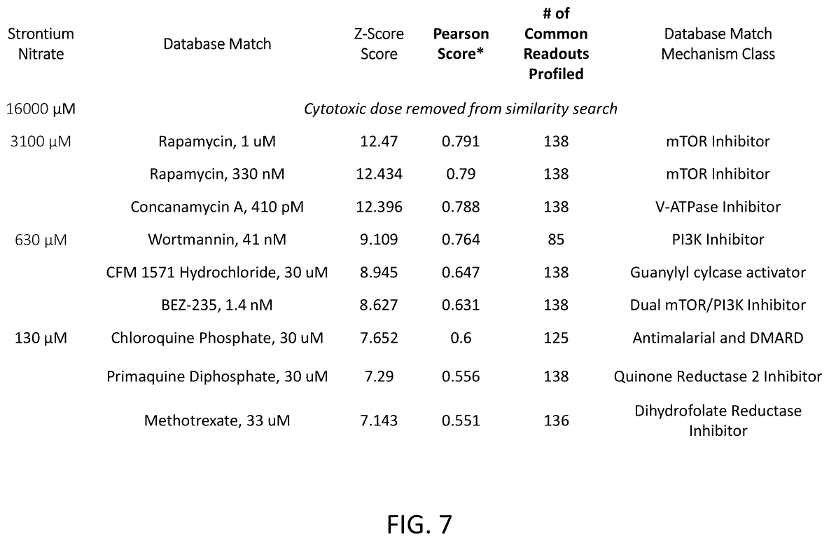

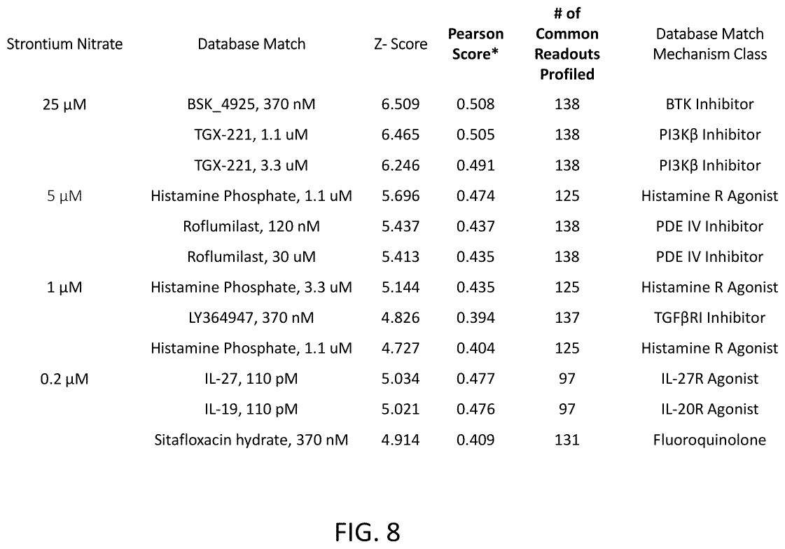

The top reference database matches for strontium nitrate are illustrated in FIGS. 7-8. In addition to rapamycin and Concanamycin A, database matches were obtained for Wortmannin (PI3K inhibitor), CFM 1571 hydrochloride (guanylyl cyclase activator), BEZ-235 (dual mTOR/PI3K inhibitor), chloroquine phosphate (antimalarial and DMARD), primaquine diphosphate (quinone reductase 2 inhibitor) and methotrexate (dihydrofolate reductase inhibitor), BSK_4925 (BTK inhibitor), TGX-221 (PI3Kβ inhibitor), histamine phosphate (histamine R agonist), roflumilast (PDE IV inhibitor), LY364947 (TGFβRI inhibitor), IL-27 (IL-27R agonist), IL-19 (IL-20R agonist) and sitafloxacin hydrate (fluoroquinoline).

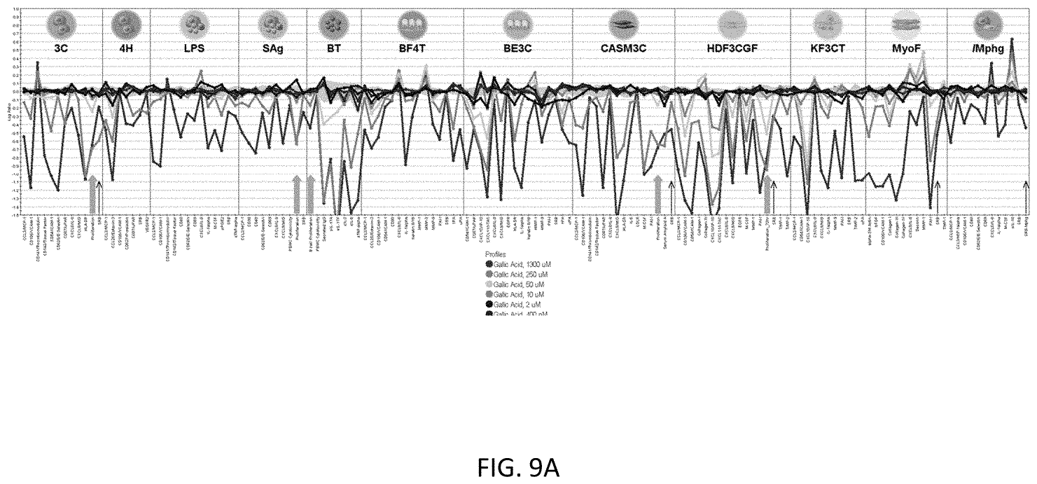

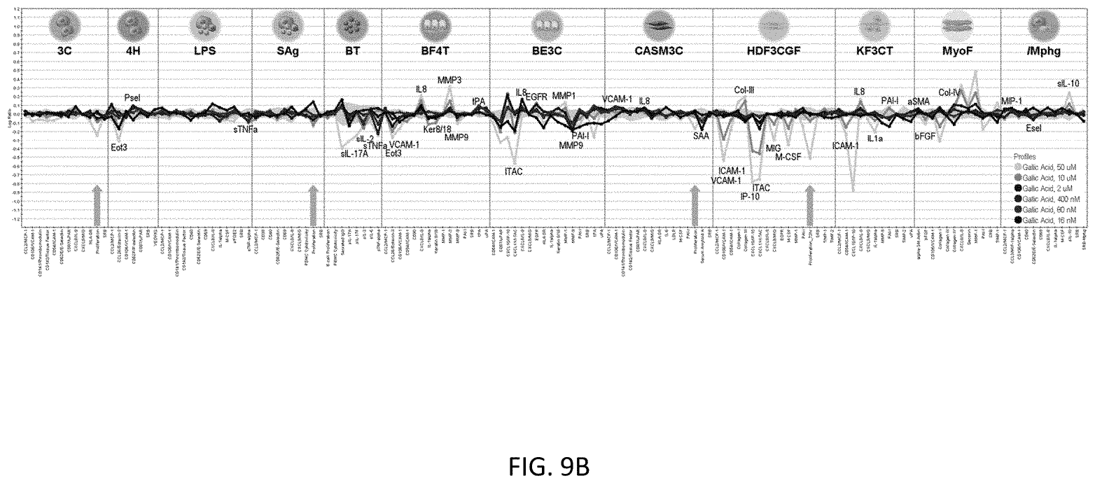

The assay profile of gallic acid at various concentrations is illustrated in FIG. 9A. The assay profile of gallic acid at non-cytotoxid concentrations is illustrated in FIG. 9B. This compound was overtly toxic in multiple systems at the top two concentrations which were removed from the analyses. As illustrated in FIG. 9, gallic acid was antiproliferative to endothelial cells, T cells, coronary artery smooth muscle cells and fibroblasts (grey arrows); affected inflammation-related activities (decreased Eotaxin-3, sTNFα, IP-10, I-TAC, SAA, ICAM-1, MIG, IL-1α and E-selectin; increased P-selectin, IL-8 and MIP-1; modulated VCAM-1); affected immunomodulatory activities (decreased sIL-17A, sIL-2 and M-CSF; increased sIL-10), affected tissue remodeling activities (decreased Keratin 8/18, MMP-9 and bFGF; increased MMP-1, MMP-3, tPA, Collagen-III, α-SMA, Collagen-IV and EGFR; modulated PAI-I). Readouts that were significantly modulated in both directions within the same system in a concentration-dependent manner are indicated by thick arrows.

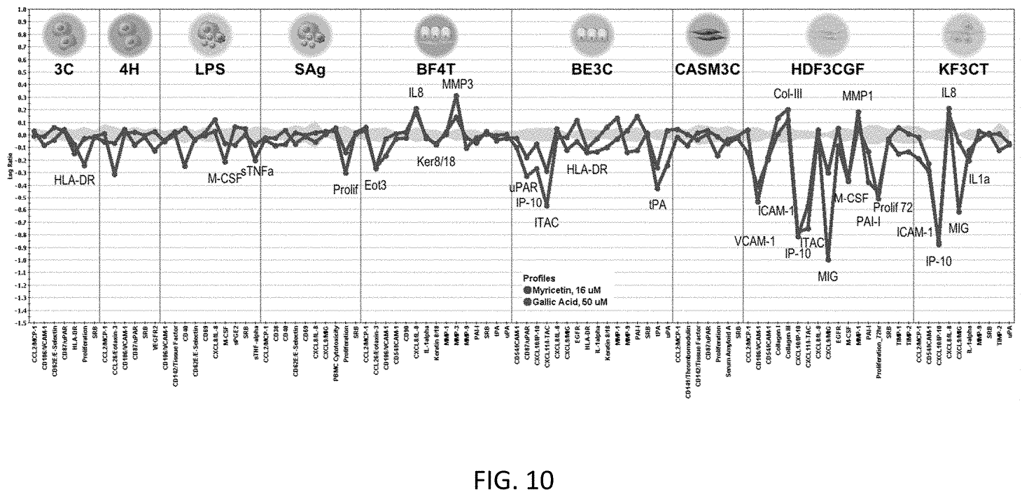

As illustrated in FIG. 10, the top database search result for gallic acid was myricetin which is a flavonoid antioxidant. An overlay of gallic acid (50 μM) and myricetin (16 μM) had a Pearson correlation r=0.739. Common activities are annotated.

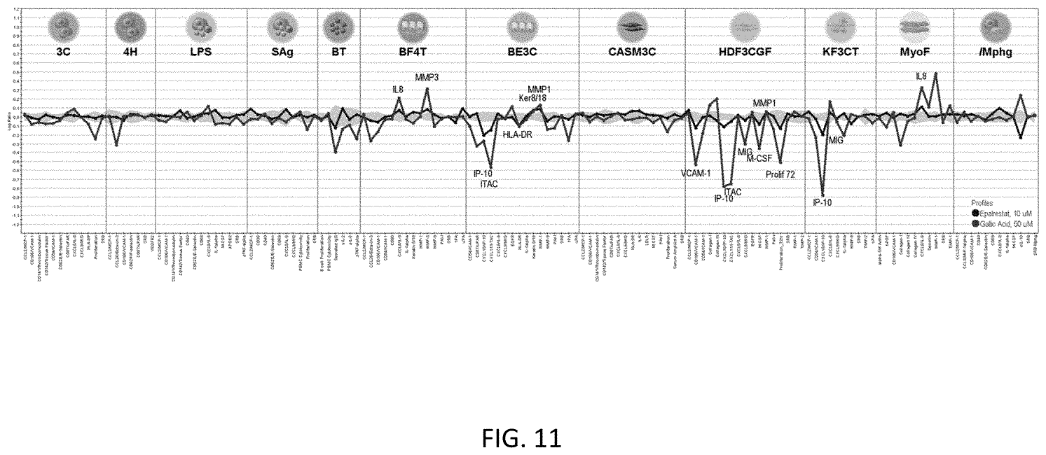

As illustrated in FIG. 11, a second database search result for gallic acid was performed. This search identified epalrestat, an aldose reductase inhibitor approved for the treatment of diabetic neuropathy. An overlay of gallic acid (50 μM) and epalrestat (10 μM) had a Pearson correlation r=0.663. The relevance of similarities at Pearson's correlations, r<0.7 is unknown. Common activities are annotated.

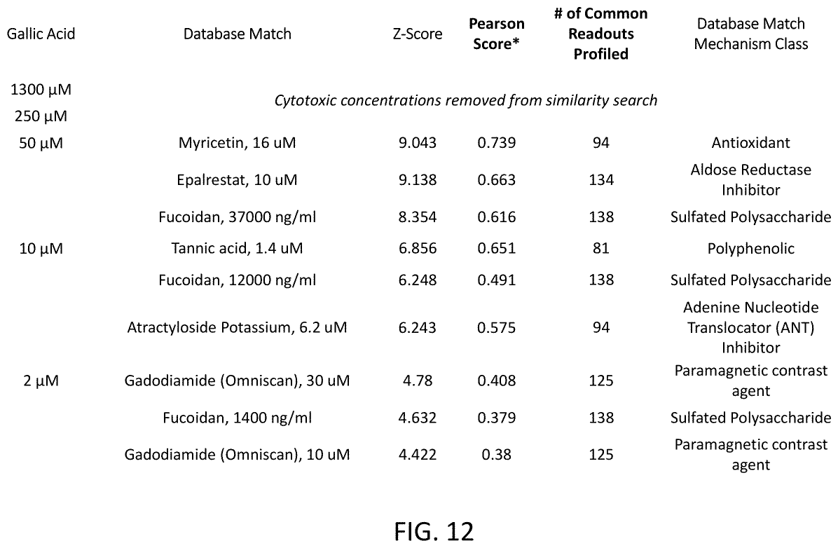

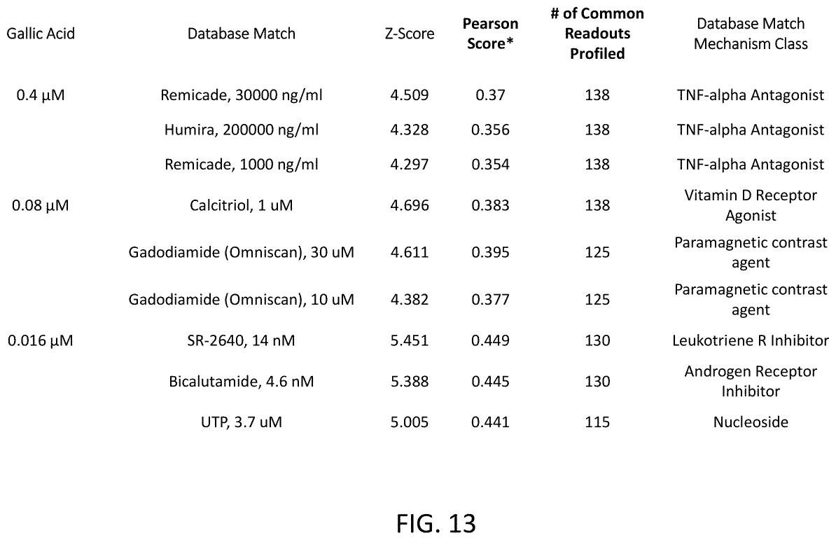

The top reference database matches for strontium nitrate are illustrated in FIGS. 12-13. In addition to myricetin and epalrestat, database matches were obtained for fucoidan (sulfated polysaccharide), tannic acid (polyphenolic), atractylosode potassium (adenine nucleotide translocator inhibitor) and gadodiamide (Omniscan) (paramagnetic contrast agent).

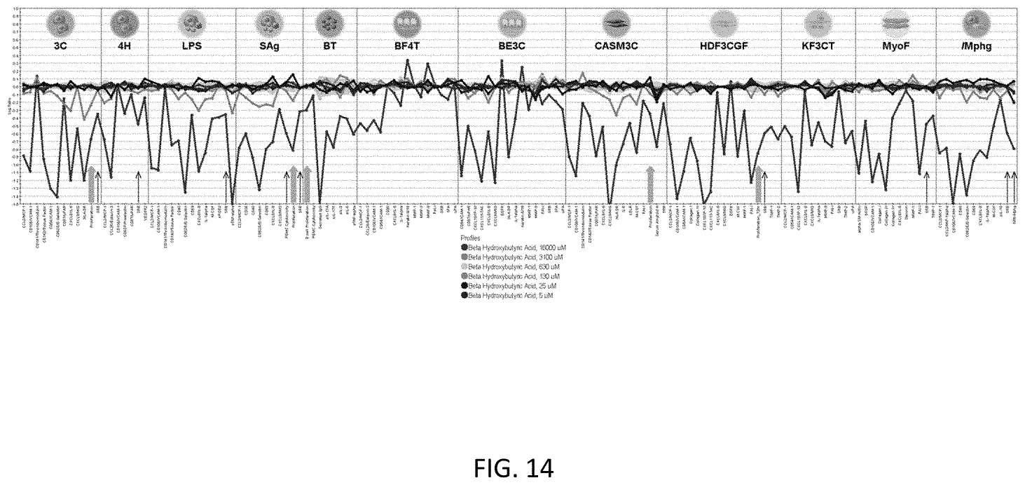

The assay profile of beta hydroxybutyric acid is illustrated in FIG. 14. This compound was overtly toxic in multiple systems at the top concentration tested which was removed from the analyses. As illustrated in FIG. 14, beta hydroxybutyric acid was anti-proliferative to endothelial and T cells (grey arrows), reduced inflammation-related activities (decreased IL-8, VCAM-1 and MIG; increased ICAM-1 and IL-1α); decreased immunomodulatory activities (decreased sIgG; increased M-CSF); and decreased tissue remodeling activities (increased tPA, uPA, EGFR and Collagen-I; modulated MMP-9 and pAI-I).

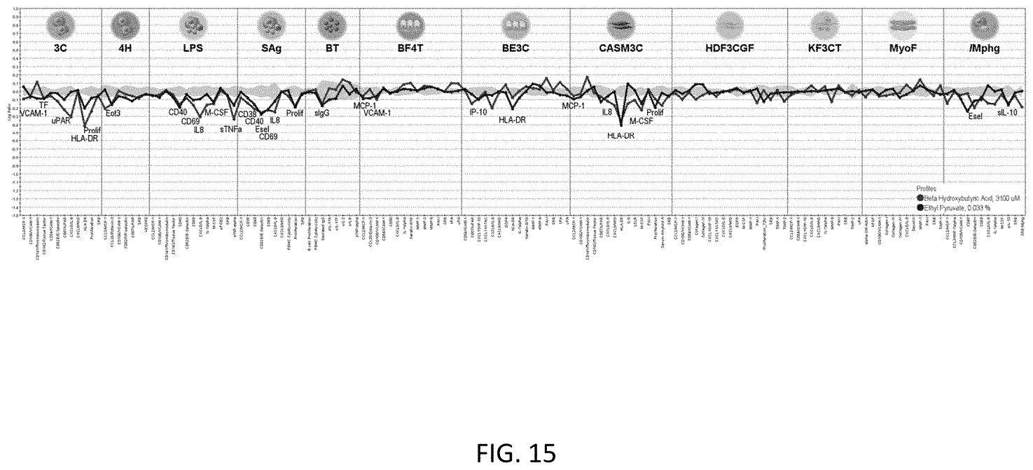

As illustrated in FIG. 15, the top database search result for beta hydroxybutyric acid was ethyl pyruvate which is a derivative of the endogenous metabolite, pyruvic acid which has anti-inflammatory and tissue-protective effects in animal models. An overlay of beta hydroxybutyric acid (3100 μM) and ethyl pyruvate (0.033%) had a Pearson correlation r=0.544. The relevance of similarities at Pearson's correlations, r<0.7 is unknown. Common activities are annotated.

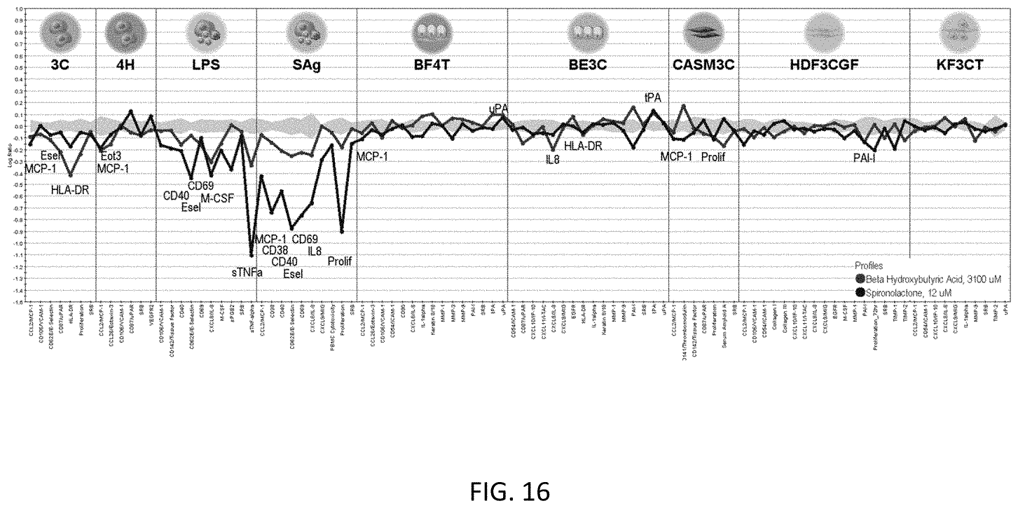

As illustrated in FIG. 16, a second database search result for beta hydroxybutyric acid was performed. This search identified spironolactone, which is an aldosterone antagonist used as a diuretic and anti-hypertensive. An overlay of beta hydroxybutyric acid (3100 μM) and spironolactone (12 μM) had a Pearson correlation r=0.569. The relevance of similarities at Pearson's correlations, r<0.7 is unknown. Common activities are annotated.

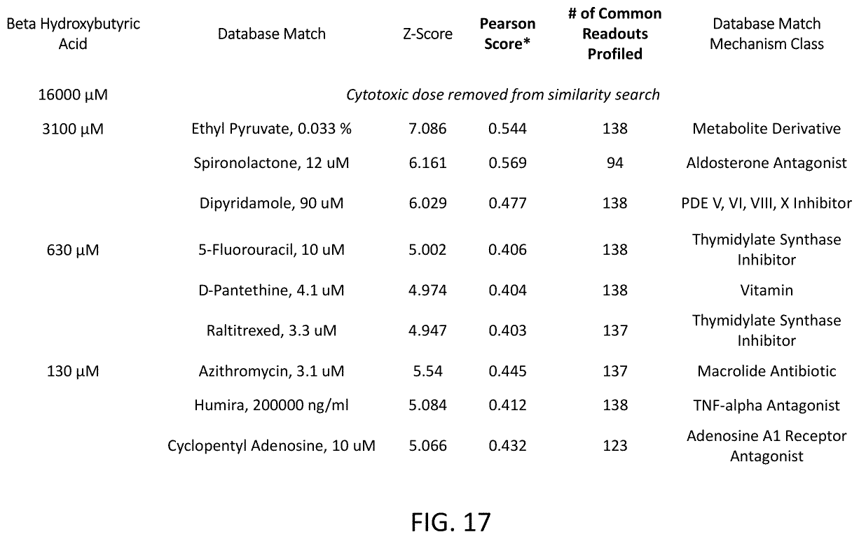

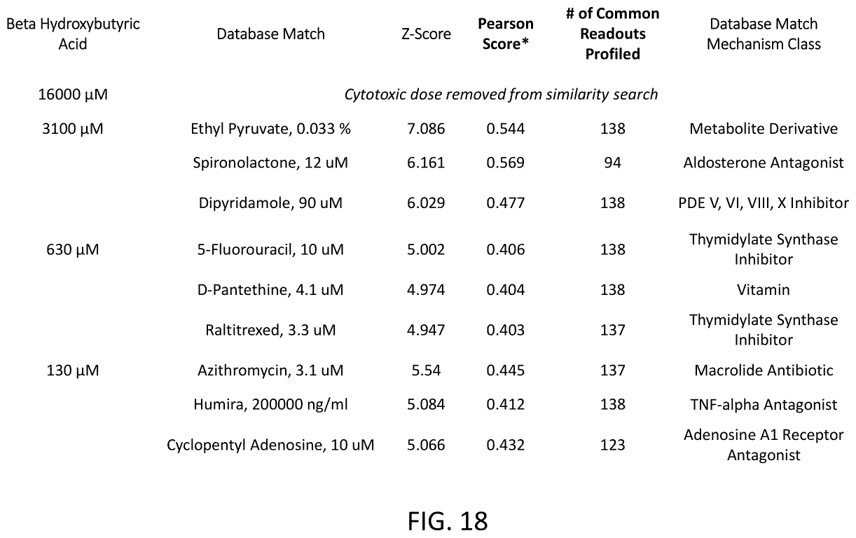

The top reference database matches for beta hydroxybutyric acid are illustrated in FIGS. 17-18. In addition to ethyl pyruvate and spironolactone, database matches were obtained for dipyridamole (PDE V, VI, VII, VIII, X inhibitor), 5-fluorouracil (thymidylate synthase inhibitor), D-Pantethine (vitamin), raltitrexed (thymidylate synthase inhibitor), azithromycin (macrolide antibiotic), Humira (TNF-α antagonist), cyclopentyl adenosine (adenosine A1 receptor antagonist), Avastin (VEGF-A inhibitor), LY364947 (TGFβR1 inhibitor), prostaglandin E2 (EP agonist), sauvagine (CRF receptor agonist), PTH(1-34) (hormone) and GRP (neuropeptide).

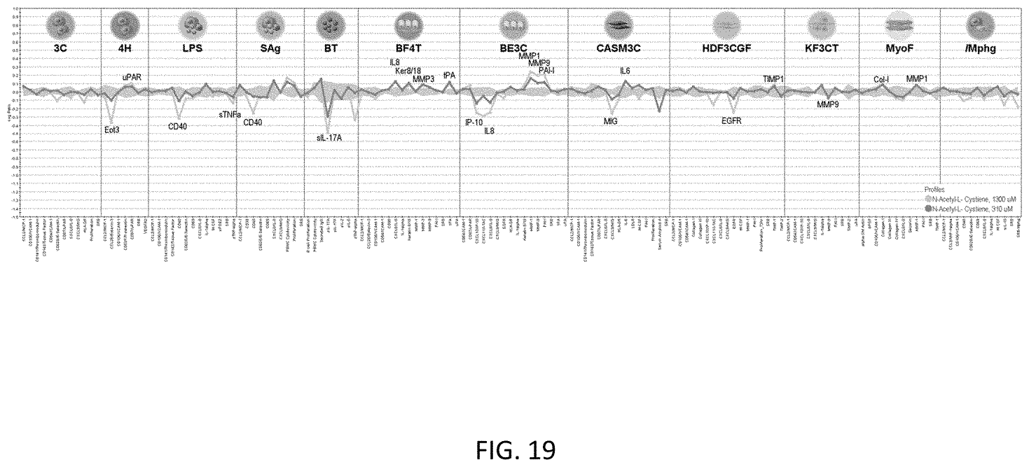

The assay profile of N-acetyl-L-cysteine is illustrated in FIG. 19. This compound was overtly toxic in multiple systems at the top two concentrations tested which were removed from the analyses. As illustrated in FIG. 19, N-acetyl-L-cysteine affected immunomodulatory activities (decreased eotaxin-3, sTNFα, IP-10 and MIG; increased IL-6; modulated IL-8); affected immunomodulatory activities (decreased CD40 and sIL-17A), and affected tissue remodeling activities (decreased EGFR; increased uPAR, Keratin 8/18, MMP-3, tPA, MMP-1, PAI-I, TIMP-I and Collagen-I; modulated MMP-9).

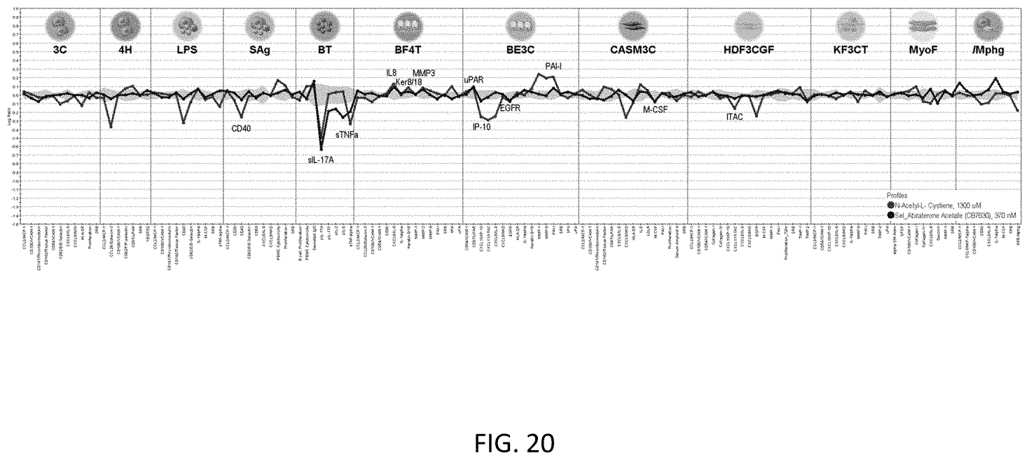

As illustrated in FIG. 20, the top database search result for N-acetyl-L-cysteine was abiraterone acetate which is a CYP P450 17A1 inhibitor. An overlay of N-acetyl-L-cysteine (1300 μM) and abiraterone acetate/CB7360 (370 nm) had a Pearson correlation r=0.453. The relevance of similarities at Pearson's correlations, r<0.7 is unknown. Common activities are annotated.

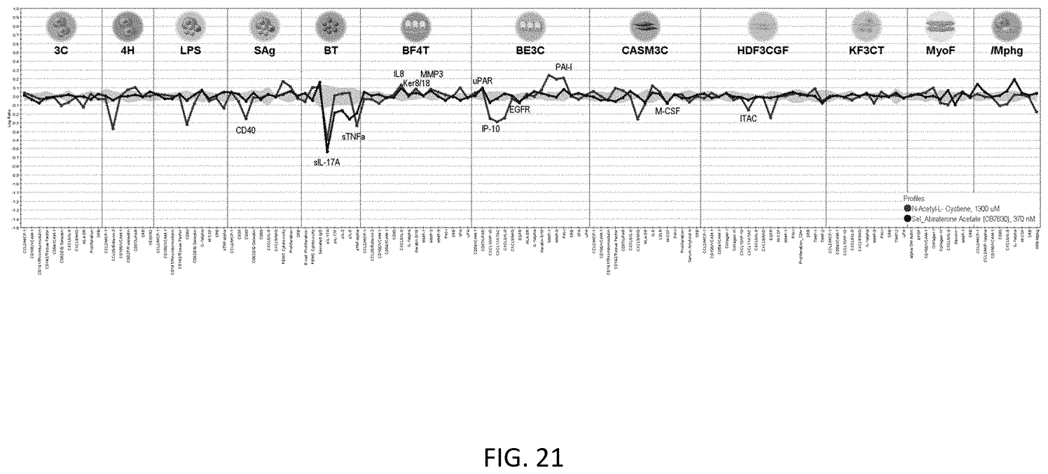

As illustrated in FIG. 21, a second database search result for N-acetyl-L-cysteine was performed. This search identified ZM 449829, which is an inhibitor of JAK3, EGFR, JAK1 and CDK4. An overlay of N-acetyl-L-cysteine (1300 μM) and ZM 449829 (14 nM) had a Pearson correlation r=0.51. The relevance of similarities at Pearson's correlations, r<0.7 is unknown. Common activities are annotated.

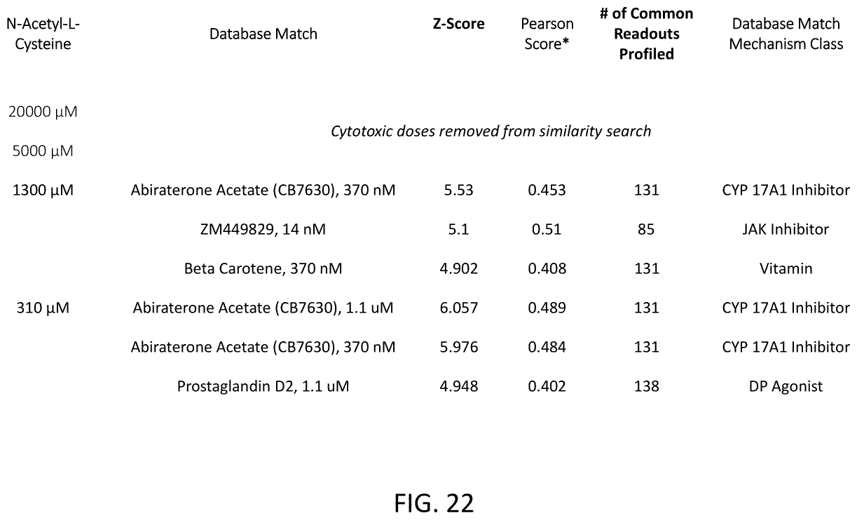

The top reference database matches for N-acetyl-L-cysteine are illustrated in FIG. 22. In addition to abiraterone acetate and ZM 449829, database matches were obtained for beta carotene (vitamin) and prostaglandin (DP agonist).

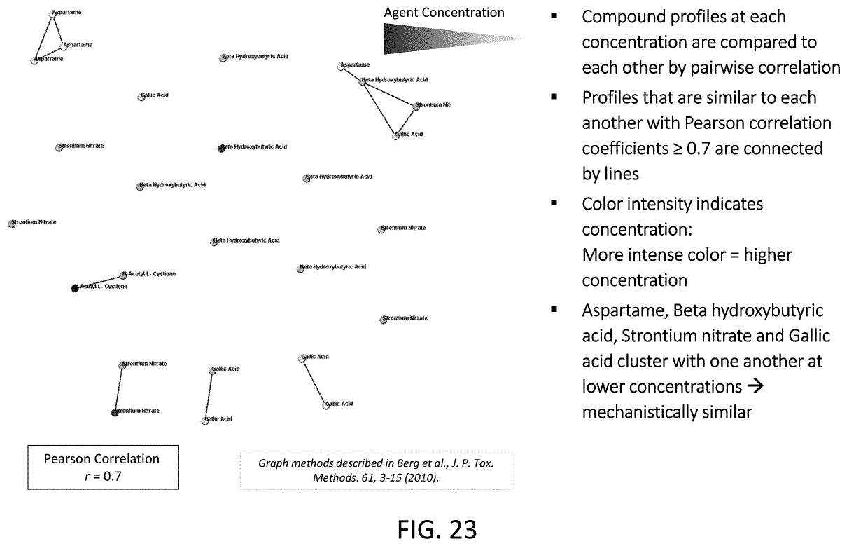

FIG. 23 illustrates the clustering of project profiles. Compound profiles at each concentration are compared to each other by pairwise correlation. Profiles that are similar to each other with Pearson correlation coefficients of at least 0.7 are connected by lines. The color intensity indicates the concentration (more intense color=higher concentration). Aspartame, beta hydroxybutyric acid, strontium nitrate and gallic acid cluster with one another at lower concentrations, and are therefore mechanistically similar.

The key responses for the test compounds are summarized as follows.

Strontium Nitrate: Anti-proliferative to endothelial cells, T cells, B cells, coronary artery smooth muscle cells and fibroblasts; Inflammation-related activities: decreased Eotaxin-3, sTNFα, VCAM-1, IL-1α, IP-10, I-TAC, IL-8, SAA, MCP-1; increased sPGE2; Immunomodulatory activities: decreased HLA-DR, sIgG, sIL-17A, sIL-17F, sIL-6; increased M-CSF; Tissue remodeling activities: decreased uPAR, MMP-1, MMP-9, PAI-I, tPA; increased MMP-3, tPA, Keratin 8/18, TIMP-2; Hemostasis-related activities: increased TM, TF.

Gallic Acid: Anti-proliferative to endothelial cells, T cells, coronary artery smooth muscle cells and fibroblasts; Inflammation-related activities: decreased Eotaxin-3, sTNFα, IP-10, I-TAC, SAA, ICAM-1, MIG, IL-1α and E-selectin; increased P-selectin, IL-8 and MIP-1; modulated VCAM-1; Immunomodulatory activities: decreased sIL-17A, sIL-2 and M-CSF; increased sIL-10; Tissue remodeling activities: decreased Keratin 8/18, MMP-9 and bFGF; increased MMP-1, MMP-3, tPA, Collagen-III, α-SMA, Collagen-IV and EGFR; modulated PAI-I.

Beta hydroxybutyric Acid: Anti-proliferative to endothelial cells and T cells; Inflammation-related activities: decreased IL-8, VCAM-1 and MIG; increased ICAM-1 and IL-1α; Immunomodulatory activities: decreased sIgG; increased M-CSF; Tissue remodeling activities: increased tPA, uPA, EGFR and Collagen-I; modulated MMP-9 and PAI-I.

N-Acetyl-L-Cysteine: Inflammation-related activities: decreased Eotaxin-3, sTNFa, IP-10 and MIG; increased IL-6; modulated IL-8; Immunomodulatory activities: decreased CD40 and sIL-17A; Tissue remodeling activities: decreased EGFR; increased uPAR, Keratin 8/18, MMP-3, tPA, MMP-1, PAI-I, TIMP-1 and Collagen-I; modulated MMP-9.

### Study CSM002-01-b

The following compounds were screened the assay system at the noted concentrations to test for anti-proliferative, inflammation-related, immunomodulatory, tissue remodeling and hemostasis-related activities at the noted concentrations (Table 3). All test compounds were dissolved in phosphate buffered saline (PBS).

TABLE 3

| | Compound | Concentrations tested |
|---|---|---|
| 1 | Strontium Nitrate | 3125 μM, 625 μM, 125 μM, 25 μM |
| 2 | Gallic Acid | 50 μM, 10 μM, 2 μM, 0.4 μM |
| 3 | Beta hydroxybutyric | 3125 μM, 625 μM, 125 μM, 25 μM |
| | Acid | |
| 4 | N-Acetyl - L- Cysteine | 1250 μM, 310 μM, 78 μM, 20 μM |

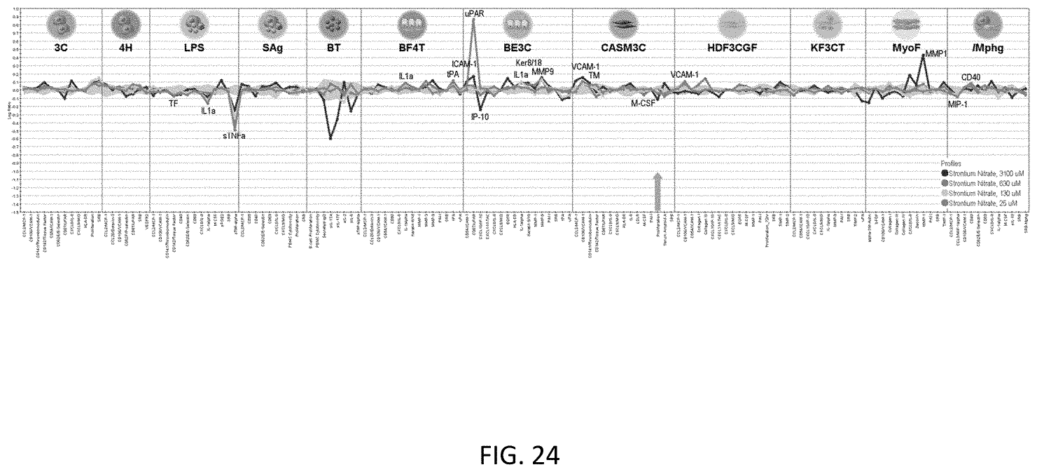

The assay profile of strontium nitrate is shown in FIG. 24. Strontium nitrate was anti-proliferative to coronary artery smooth muscle cells (grey arrow). This compound also affected inflammation-related activities (decreased sTNFα, IP-10 and MIP-1, and increased ICAM-1 and VCAM-1; modulated IL1α); affected immunomodulatory activities (decreased M-CSF; increased CD40); affected tissue remodeling activities (increased tPA, uPAR, Ker 8/18, MMP-9 and MMP-1)); and affected hemostatis-related activities (increased TM, decreased TF).

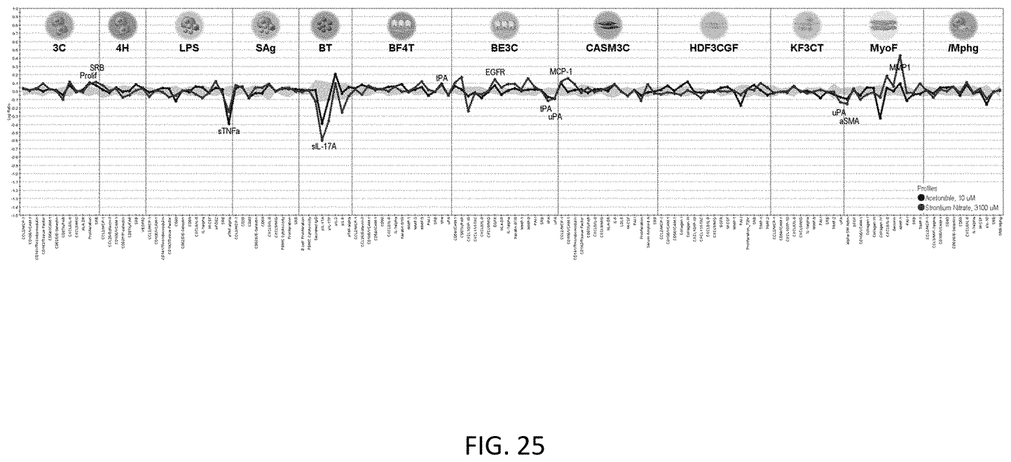

As illustrated in FIG. 25, the top database search result for strontium nitrate was acetonitrile, a solvent used in the purification of butadiene. An overlay of strontium nitrate (3100 μM) and acetonitrile (10 μM) had a Pearson correlation r=0.671. Common activities are annotated. The similarity is likely due to the common biology affected.

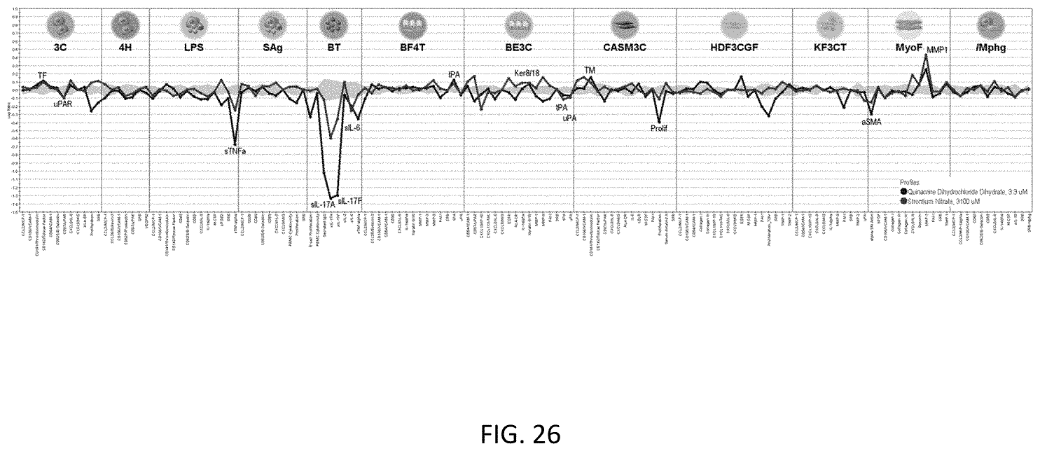

As illustrated in FIG. 26, a second database search result for strontium nitrate was performed. This search identified quinacrine dihydrochloride dihydrate which is a phospholipase A2 inhibitor. An overlay of strontium nitrate (3100 μM) and quinacrine dihydrochloride dihydrate (3.3 μM) had a Pearson correlation r=0.663. Common activities are annotated. The similarity is likely due to the common biology affected.

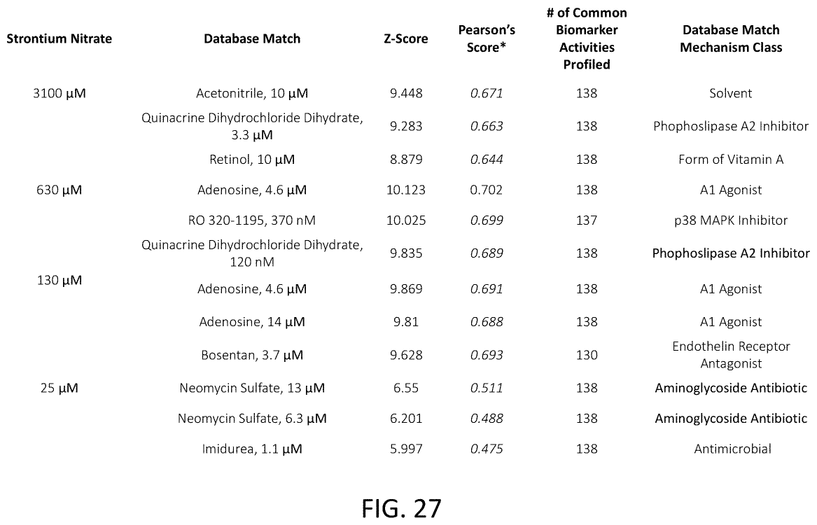

The top reference database matches for strontium nitrate are illustrated in FIG. 27. In addition to acetonitrile and quinacrine dihydrochloride dihydrate, database matches were obtained for retinol (form of vitamin A), adenosine (A1 agonist), RO 320-1195 (p38 MAPK inhibitor), bosentan (endothelin receptor antagonist), neomycin sulfate (aminoglycoside antibiotic) and imidurea (antimicrobial).

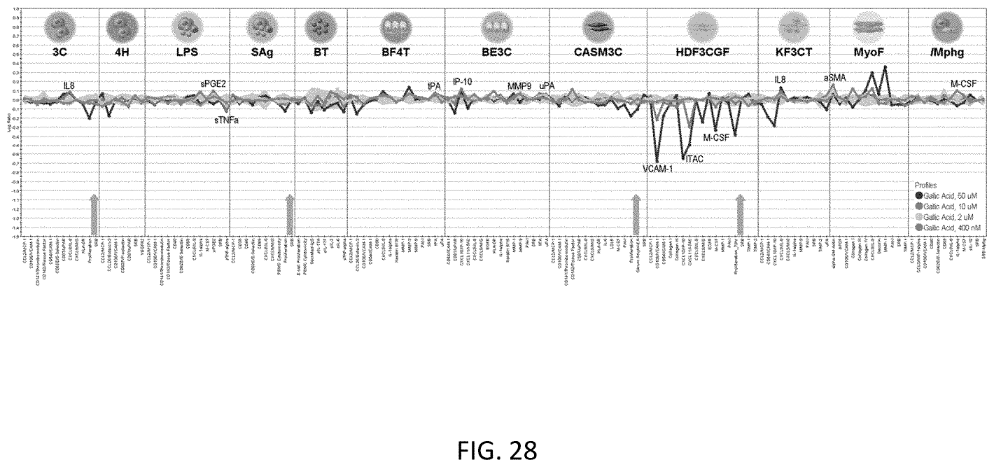

The assay profile of gallic acid is shown in FIG. 28. Gallic acid was anti-proliferative to endothelial cells, T cells, coronary artery smooth muscle cells and fibroblasts (grey arrows). This compound also affected inflammation-related activities (decreased sTNFα, VCAM-1, and ITAC; increased IL-8, sPGE₂ and IP-10); affected immunomodulatory activities (modulated M-CSF); and affected tissue remodeling activities (increased tPA, MMP-9, uPA and αSMA).

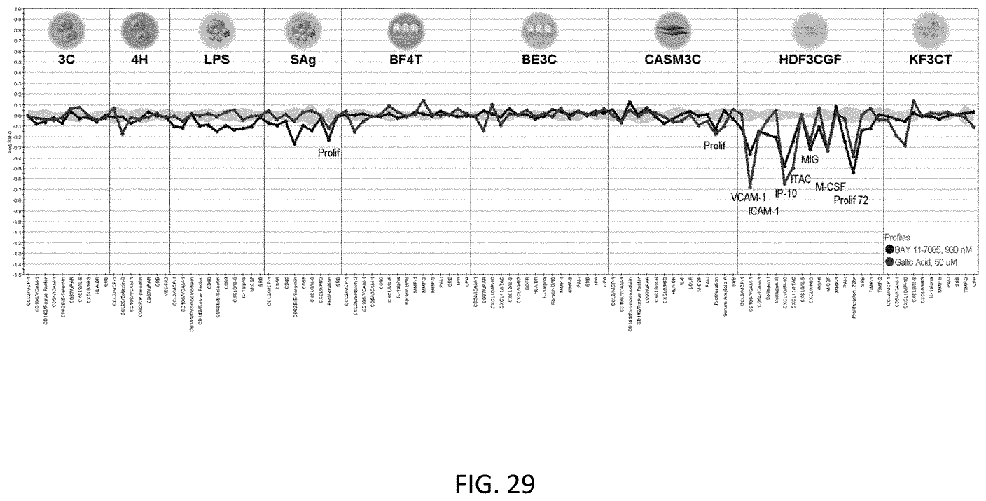

As illustrated in FIG. 29, the top database search result for gallic acid was BAY 11-7085, an irreversible inhibitor of IκBα phosphorylation, preventing activation of NF-κB. An overlay of gallic acid (50 μM) and BAY 11-7085 (930 nM) had a Pearson correlation r=0.694. Common activities are annotated. The similarity is likely due to the common biology affected.

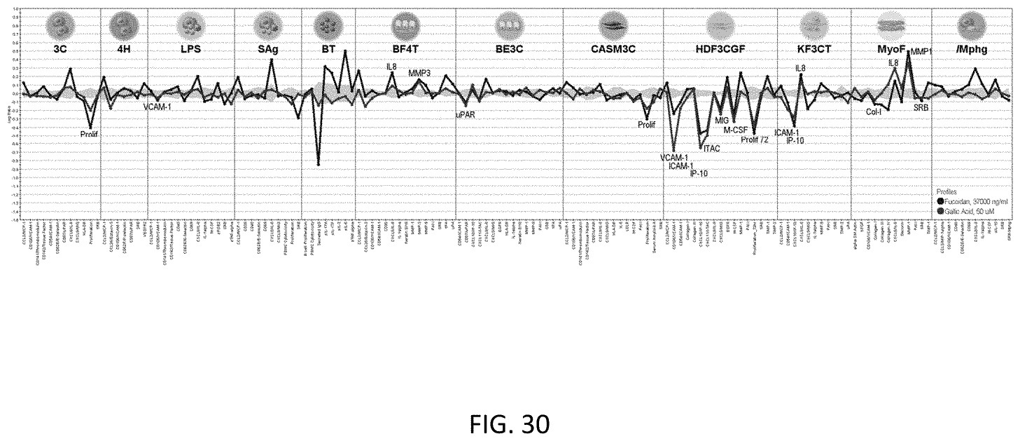

As illustrated in FIG. 30, a second database search result for gallic acid was performed. This search identified fucoidan which is a L-selectin antagonist found in various species of algae and seaweed. An overlay of gallic acid (50 μM) and fucoidan (3700 ng/ml) had a Pearson correlation r=0.636. Common activities are annotated. The similarity is likely due to the common biology affected.

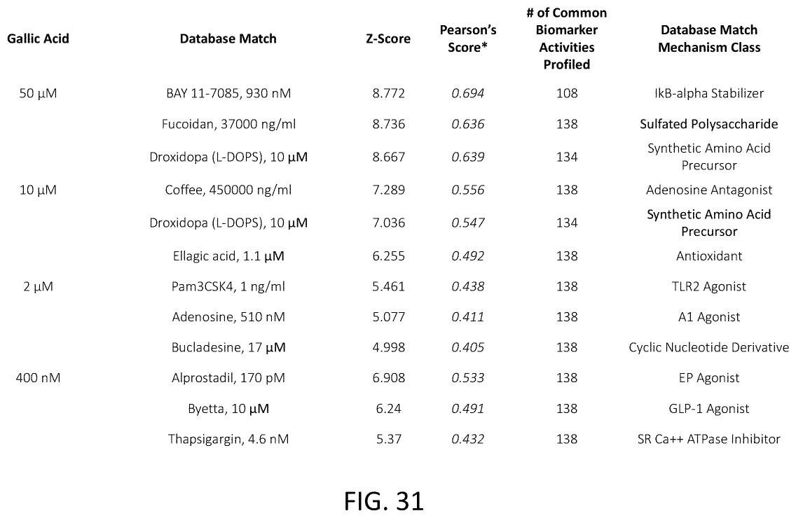

The top reference database matches for strontium nitrate are illustrated in FIG. 31 In addition to BAY 11-7085 and fucoidan, database matches were obtained for drixidopa (L-DOPS) (synthetic amino acid precursor), coffee (adenosine antagonist), ellagic acid (antioxidant), Pam3CSK4 (TLR2 agonist), adenosine (A1 agonist), bucladesine (cyclic nucleotide derivative), alprostadil (EP agonist), Byetta (GLP-1 agonist) and thapsigargin (SR Ca++ ATPase inhibitor).

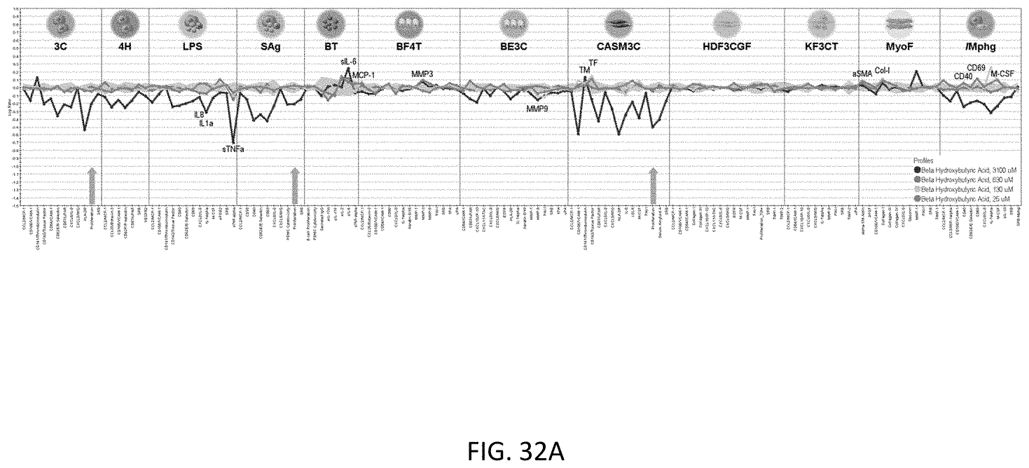

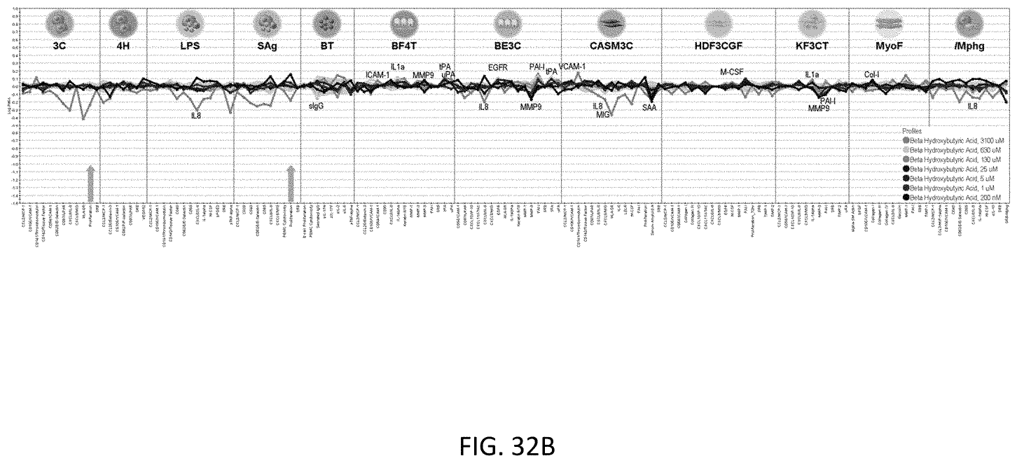

A first assay profile of beta hydroxybutyric acid is shown in FIG. 32A. A second assay profile of beta hydroxybutyric acid is shown in FIG. 32B. Beta hydroxybutyric acid was anti-proliferative to endothelial cells, T cells and coronary artery smooth muscle cells (grey arrows). This compound also affected inflammation-related activities (decreased IL-8, IL1α and sTNFα; increased MCP-1); affected immunomodulatory activities (increased sIL-6, CD40, CD69 and M-CSF); affected tissue remodeling activities (increased MMP-3, αSMA and Col-I; decreased MMP9); and affected hemostasis-related activities (increased TM and TF).

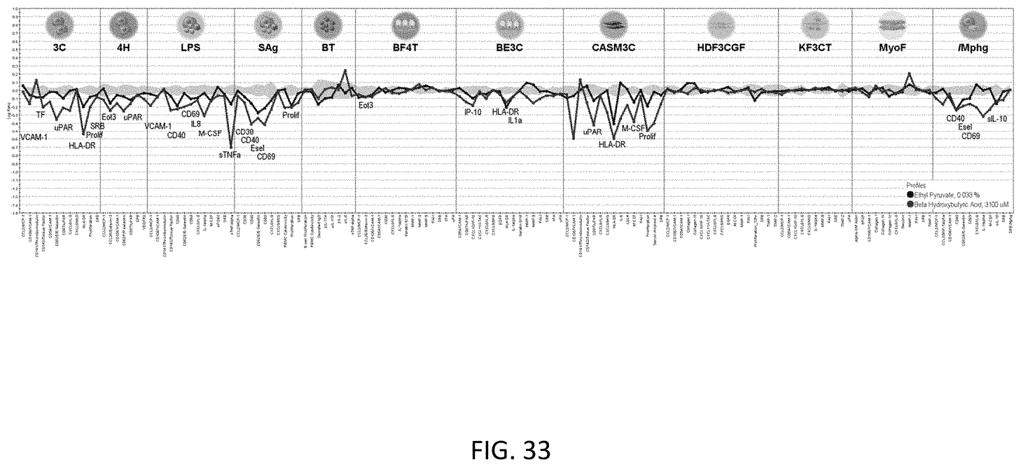

As illustrated in FIG. 33, the top database search result for beta hydroxybutyric acid was ethyl pyruvate, a compound which inhibits the systemic release of cytokines, such as TNF-α and HMGB 1. An overlay of beta hydroxybutyric acid (3100 μM) and ethyl pyruvate (0.033%) had a Pearson correlation r=0.578. Common activities are annotated. The similarity is likely due to the common biology affected.

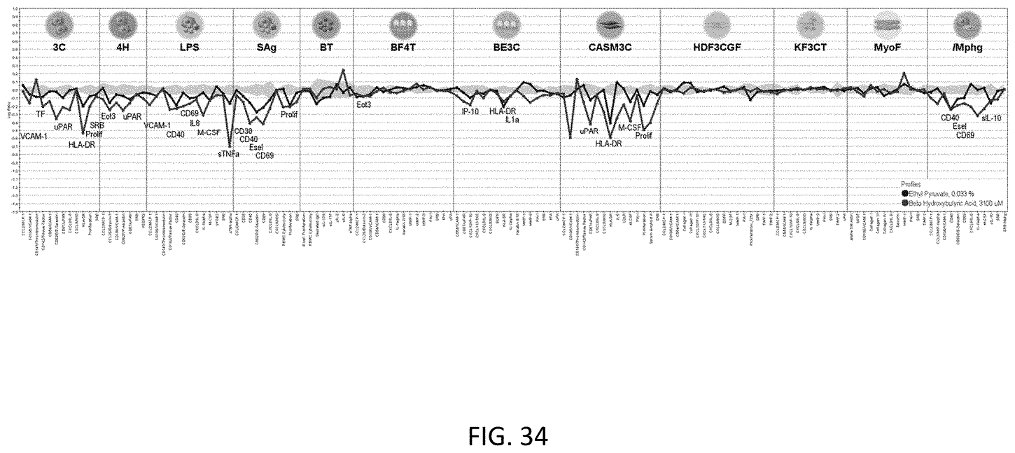

As illustrated in FIG. 34, a second database search result for beta hydroxybutyric acid was performed. This search identified Go 6976 which is a selective inhibitor of protein kinase C and a potent antagonist of HIV-1. An overlay of beta hydroxybutyric acid (3100 μM) and Go 6976 (3 μM) had a Pearson correlation r=0.587 Common activities are annotated. The similarity is likely due to the common biology affected.

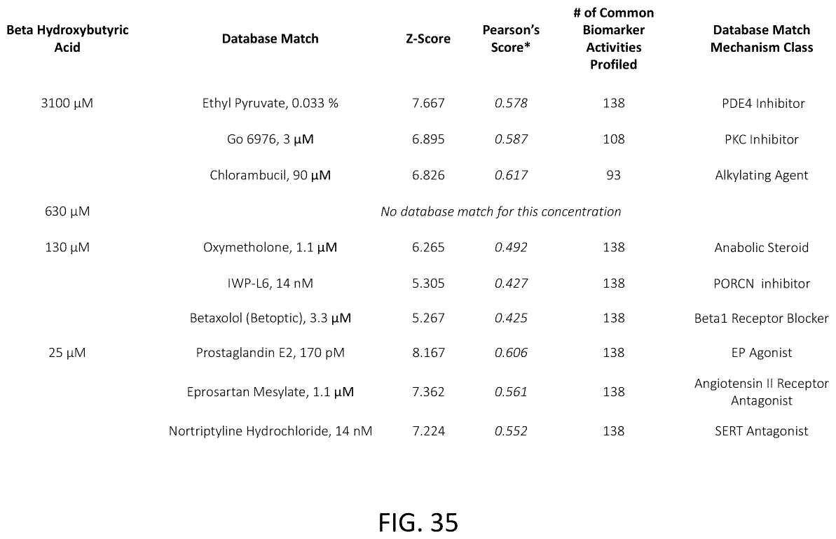

The top reference database matches for beta hydroxybutyric acid are illustrated in FIG. 35. In addition to ethyl pyruvate and Go 6976, database matches were obtained for chlorambucil (alkylating agent), oxymrthylone (anabolic steroid), IWP-L6 (PORCN inhibitor), Betaxolol (betoptic) (beta1 receptor blocker), prostaglandin E2 (EP agonist), eprosartan mesylate (angiotensin II receptor antagonist) and nortriptyline hydrochloride (SERT antagonist).

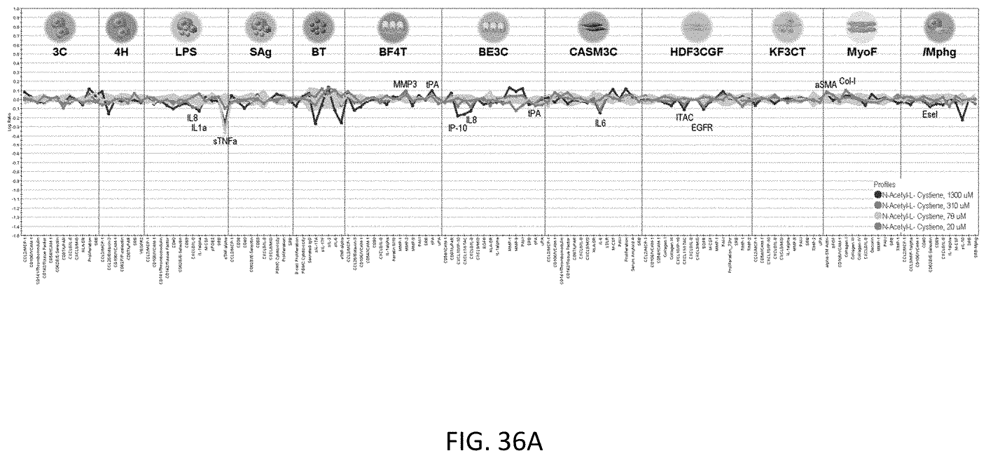

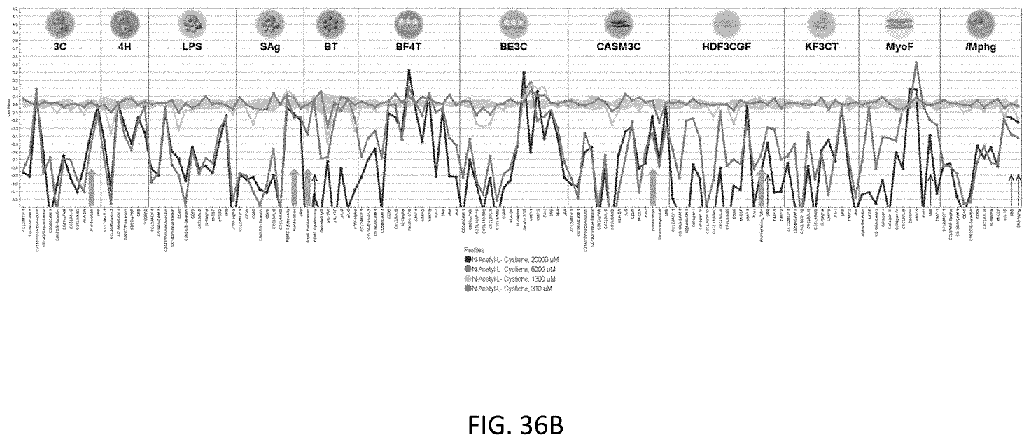

A first assay profile of N-acetyl-L-cysteine is shown in FIG. 36A. A second assay profile of N-acetyl-L-cysteine is shown in FIG. 36B. N-acetyl-L-cysteine was affected inflammation-related activities (decreased IL-8, IL1α, sTNFα, IP-10, IL-6, ITAC and Esel); and affected tissue remodeling activities (decreased EGFR; increased MMP3, αSMA and Col-1; modulated tPA).

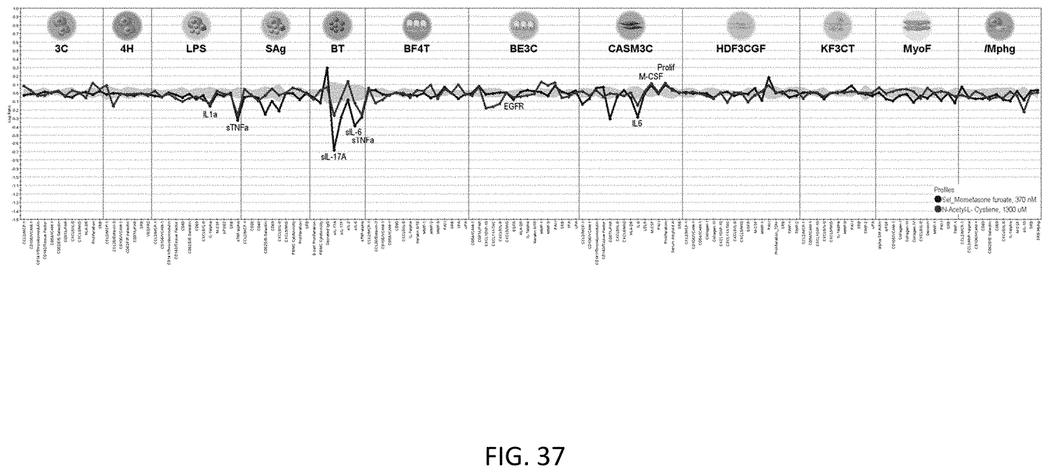

As illustrated in FIG. 37, the top database search result for N-acetyl-L-cysteine was mometasone furoate, a compound which inhibits cytochrome P450 2C8. An overlay N-acetyl-L-cysteine (1300 μM) and mometasone furoate (370 nM) had a Pearson correlation r=0.495. Common activities are annotated. The similarity is likely due to the common biology affected.

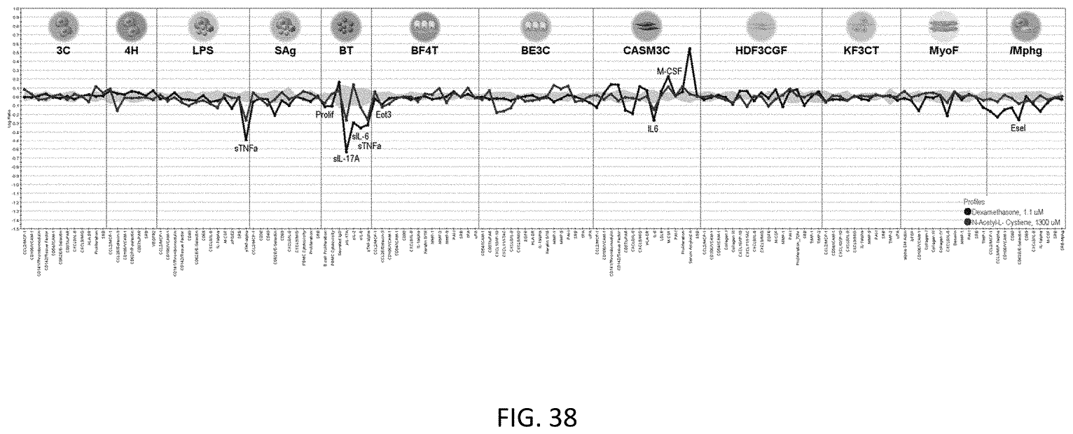

As illustrated in FIG. 38, a second database search result for N-acetyl-L-cysteine was performed. This search identified dexamethasone which is a glucocorticoid receptor agonist used to treat many inflammatory and autoimmune conditions. An overlay of N-acetyl-L-cysteine (1300 μM) and dexamethasone (1.1 μM) had a Pearson correlation r=0.481. Common activities are annotated. The similarity is likely due to the common biology affected.

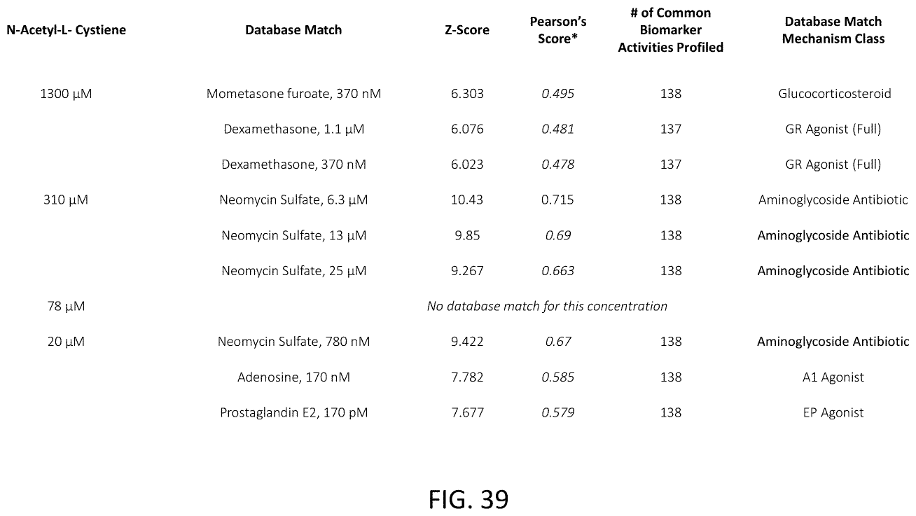

The top reference database matches for N-acetyl-L-cysteine are illustrated in FIG. 39. In addition to mometasone dexamethasone and dexamethasone, database matches were obtained for neomycin sulfate (aminoglycoside antibiotic), adenosine (A1 agonist) and prostaglandin E2 (EP agonist).

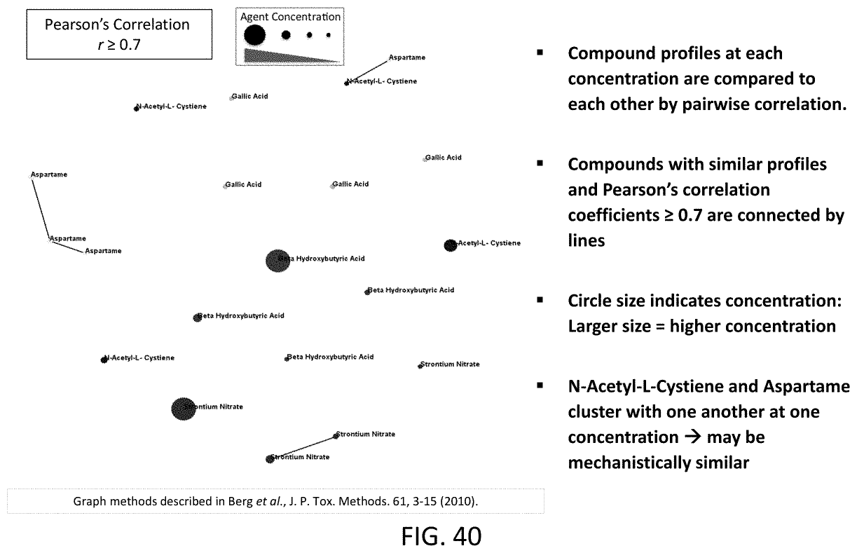

FIG. 40 illustrates the clustering of project profiles. Compound profiles at each concentration are compared to each other by pairwise correlation. Profiles that are similar to each other with Pearson correlation coefficients of at least 0.7 are connected by lines. The color intensity indicates the concentration (more intense color=higher concentration). Aspartame, beta hydroxybutyric acid, strontium nitrate and gallic acid cluster with one another at lower concentrations, and are therefore mechanistically similar.

The key responses for the test compounds are summarized as follows.

Strontium Nitrate: Anti-proliferative to coronary artery smooth muscle cells; Inflammation-related activities: decreased sTNFα, IP-10, and MIP-1; increased ICAM-1 and VCAM-1; modulated IL1α; Immunomodulatory activities: decreased M-CSF; increased CD40; Tissue remodeling activities: increased tPA, uPAR, Ker8/18, MMP9, and MMP1; Hemostasis-related activities: decreased TF; increased TM.

Gallic Acid: Anti-proliferative to endothelial cells, T cells, coronary artery smooth muscle cells, and fibroblasts; Inflammation-related activities: decreased sTNFα, VCAM-1, and ITAC; increased IL8, sPGE₂, and IP-10; Immunomodulatory activities: modulated M-CSF; Tissue remodeling activities: increased tPA, MMP9, uPA, and αSMA.

Beta hydroxybutyric Acid: Anti-proliferative to endothelial cells, T cells, and coronary artery smooth muscle cells; Inflammation-related activities: decreased IL8, IL1α, and sTNFα; increased MCP-1; Immunomodulatory activities: increased sIL-6, CD40, CD69, and M-CSF; Tissue remodeling activities: decreased MMP9; increased MMP3, αSMA, and Col-I; Hemostasis-related activities: increased TM and TF.

N-Acetyl-L-Cysteine: Inflammation-related activities: decreased IL8, IL1α, sTNFα, IP-10, IL6, ITAC, and Esel; Tissue remodeling activities: decreased EGFR; increased MMP3, αSMA, and Col-I; modulated tPA.

### Study CSM003-01-b

The following compounds were screened in the assay system at the noted concentrations to test for anti-proliferative, inflammation-related, immunomodulatory, tissue remodeling and hemostasis-related activities at the noted concentrations (Table 4). All test compounds were dissolved in phosphate buffered saline (PBS).

TABLE 4

| | Compound | Concentrations tested |
|---|---|---|
| 1 | Strontium Nitrate, | 3100 μM, 630 μM, 130 μM, 25 μM |
| | anhydrous | |
| 2 | Strontium Chloride | 3100 μM, 630 μM, 130 μM, 25 μM |
| | hexahydrate | |
| 3 | Formula 787 | 1.0%, 0.2%, 0.04%, 0.008% |
| | (Strontium Chloride + | |
| | Gallic Acid + | |
| | N-Acetyl-L- Cysteine, | |
| | in equimolar conc.) | |
| 4 | Formula F11b | 1.0%, 0.2%, 0.04%, 0.008% |
| | (Strontium hydroxide + | |
| | Beta hydroxybutyrate + | |
| | N-Acetyl-L- Cysteine | |
| | in equimolar conc.) | |

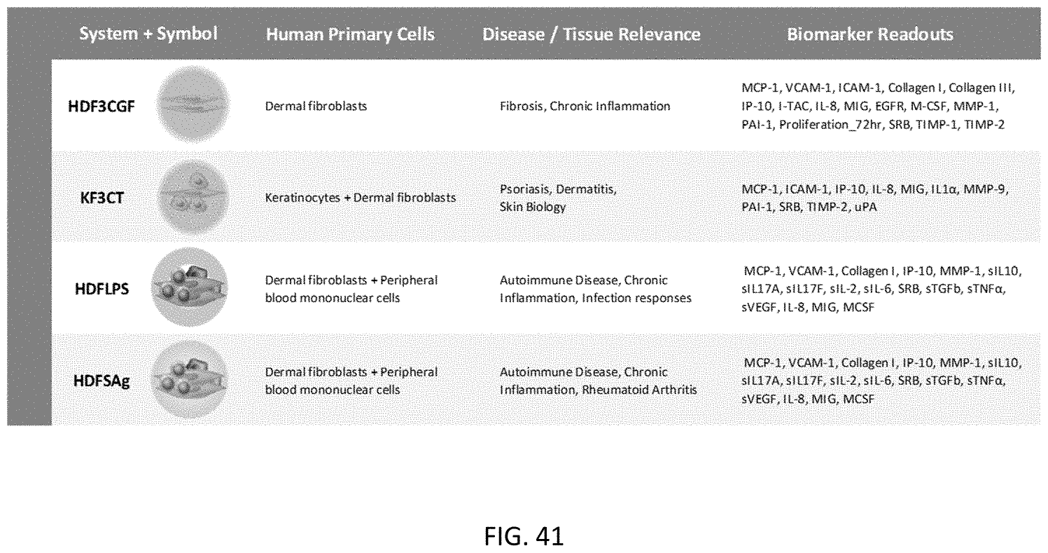

The following primary human cells were used as test systems: HDFSAg (dermal fibroblasts), HDF3CGF (dermal fibroblasts); KF3CT (keratinocytes+dermal fibroblasts) and HDFLPS (dermal fibroblasts+peripheral blood mononuclear cells). The disease/tissue relevance and biomarker endpoints for these systems is shown in FIG. 41.

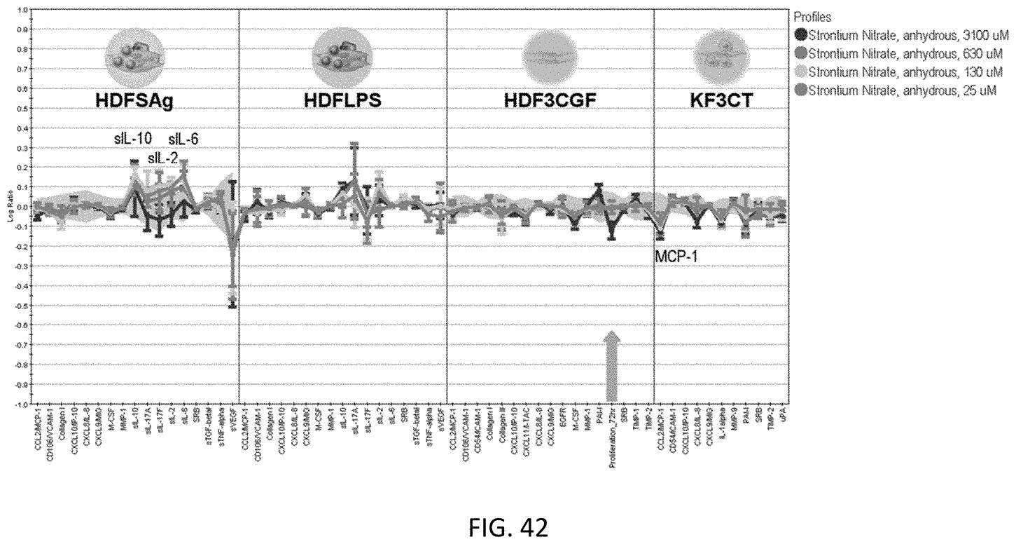

The assay profile for strontium nitrate, anhydrous is illustrated in FIG. 42. Biomarkers were annotated when values were significantly different from vehicle controls (p<0.01), are outside of the envelope and have an effect size of at least 20%. Strontium nitrate, anhydrous was anti-proliferative to fibroblasts at the top concentration only (grey arrow). This compound affected inflammation-related activities (decreased MCP-1) and affected immunomodulatory activities (increased sIL-10, sIL-2 and sIL-6). This compound exhibited an interesting pattern of enhanced immune responses in HDFSAg that may be relevant for immune oncology.

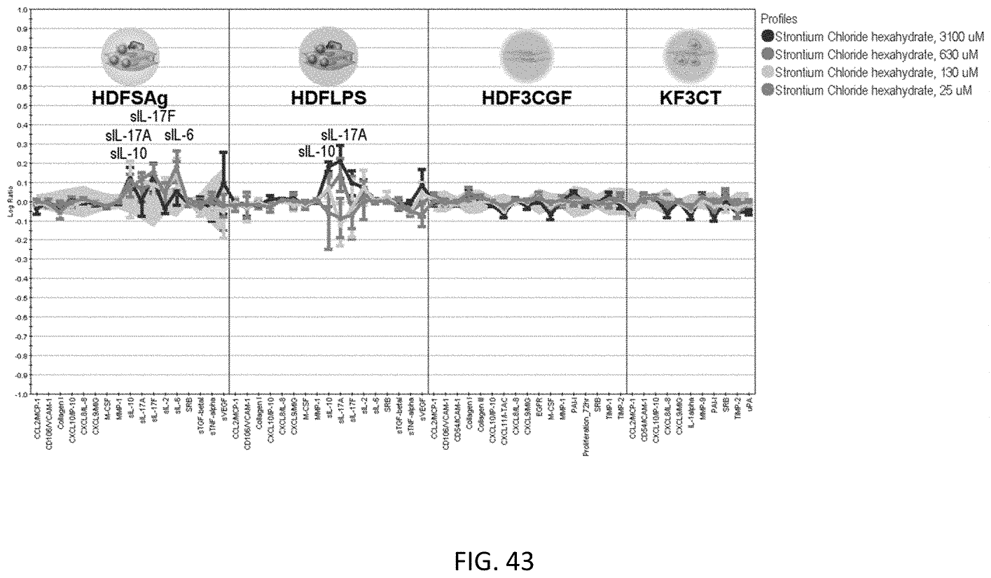

The assay profile for strontium chloride hexahydrate is illustrated in FIG. 43. Biomarkers were annotated when values were significantly different from vehicle controls (p<0.01), are outside of the envelope and have an effect size of at least 20%. Strontium chloride hexahydrate affected immunomodulatory activities (increased sIL-10, sIL-17A, sIL-17F and sIL-6). This compound exhibited an interesting pattern of enhanced immune responses in HDFSAg that may be relevant for immune oncology.

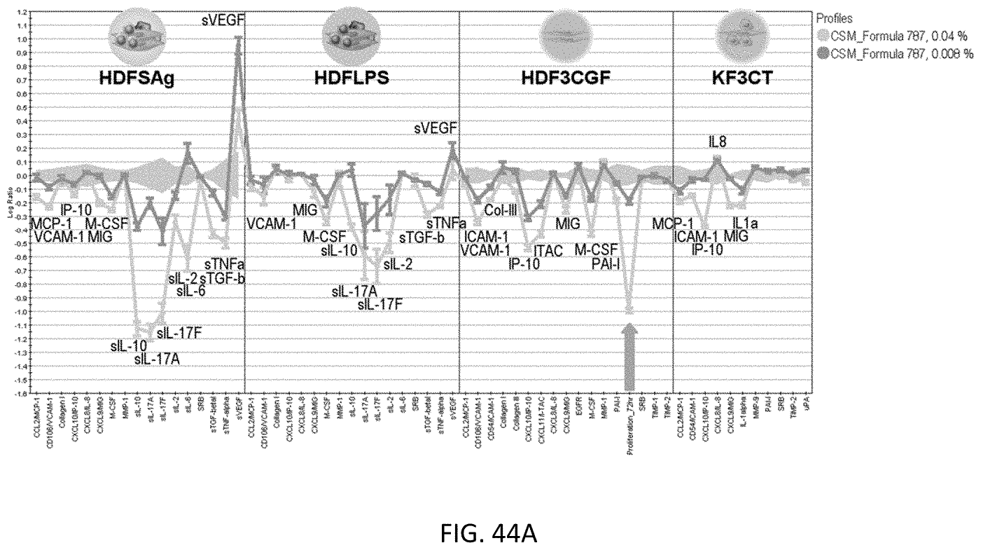

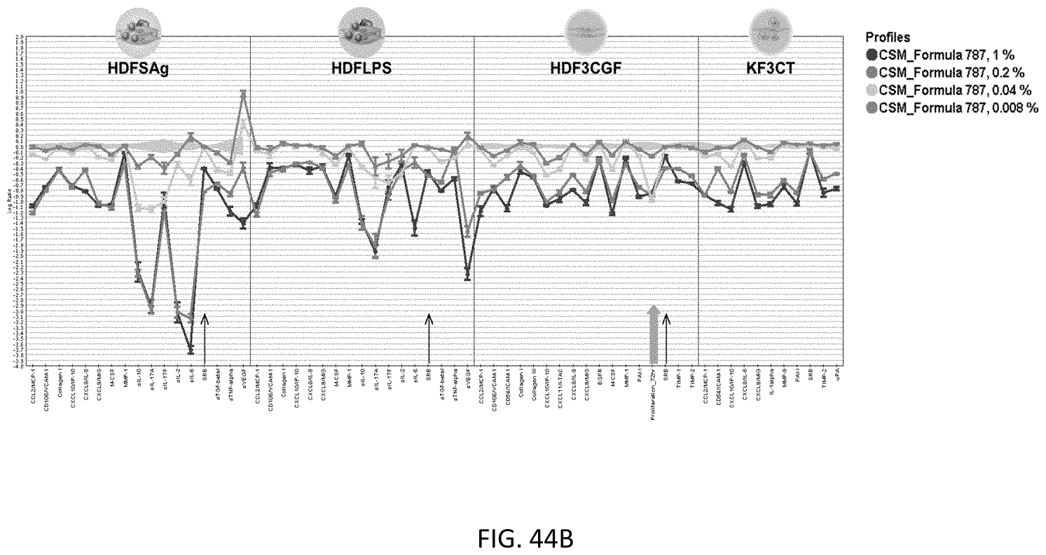

Formula 787 is broadly cytotoxic in HDFSAg, HDFLPS and HDF3CGF systems (thin black arrows) at both 1% and 0.2%. Cytotoxic concentrations were excluded from the annotation and analysis. The assay profile of Formula 787 at non-cytotoxic concentrations is illustrated in FIG. 44A. The assay profile of Formula 787 at various concentrations is illustrated in FIG. 44B. Biomarkers were annotated when values were significantly different from vehicle controls (p<0.01), are outside of the envelope and have an effect size of at least 20%. Strontium nitrate, anhydrous was anti-proliferative to fibroblasts (grey arrow). This formula affected inflammation-related activities (decreased VCAM-1, sTNFα, ICAM-1, IP-10, ITAC, MIG, MCP-1 and IL-1α; increased IL-8), immunomodulatory activities (decreased M-CSF, sIL-10, sIL-17A, sIL-17F, and sIL-2; concentration-dependent effects were observed with sIL-6 in HDFSAg) and tissue remodeling activities (decreased Col-III, PAI-I and sTGF-β; increased sVEGF).

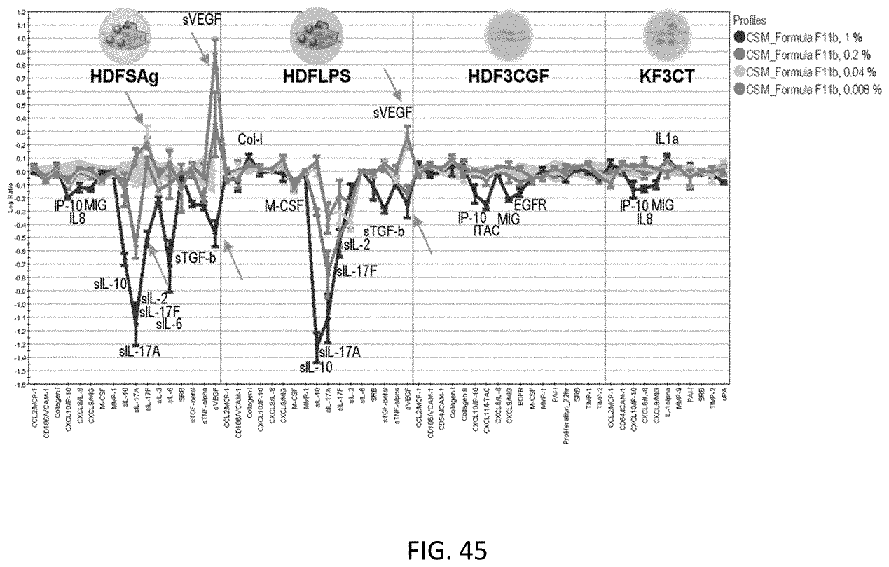

The assay profile of Formula F11b at is illustrated in FIG. 45. Biomarkers were annotated when values were significantly different from vehicle controls (p<0.01), are outside of the envelope and have an effect size of at least 20%. This formula affected inflammation-related activities (decreased IP-10, IL-8, MIG, and ITAC; increased IL-1α), immunomodulatory activities (decreased sIL-10, sIL-17A, sIL-2, sIL-6, sIL-17F and M-CSF; concentration-dependent effects were observed with sIL-17F in HDFSAg) and tissue remodeling activities (decreased EGFR and sTGF-b; increased Col-1; concentration-dependent effects observed with sVEGF in HDFSAg and HDFLPS). The number of annotated biomarkers for strontium nitrate, anhydrous, strontium chloride hexahydrate, Formula 787, and Formula F11b was 4, 6, 37, and 26, respectively.

The key responses for test compounds are summarized as follows.

Strontium Nitrate: Inflammation-related activities: decreased MCP-1; Immunomodulatory activities: increased sIL-10, sIL-2, and sIL-6; Interesting pattern of enhanced immune responses in HDFSAg that may be relevant for immune oncology.

Strontium Chloride: Immunomodulatory activities: increased sIL-10, sIL-17A, sIL-17F and sIL-6; Interesting pattern of enhanced immune responses in HDFSAg that may be relevant for immune oncology.

Formula 787: Anti-proliferative to fibroblasts; Inflammation-related activities: decreased VCAM-1, sTNFα, ICAM-1, IP-10, ITAC, MIG, MCP-1, and IL-1α; increased IL-8; Immunomodulatory activities: decreased M-CSF, sIL-10, sIL-17A, sIL-17F, and sIL-2; concentration-dependent effects observed with sIL-6 in HDFSAg; Tissue remodeling activities: decreased Col-III, PAI-I, and sTGF-β; increased sVEGF.

Formula F11b: Inflammation-related activities: decreased IP-10, IL-8, MIG, and ITAC; increased IL-1α; Immunomodulatory activities: decreased sIL-10, sIL-17A, sIL-2, sIL-6, sIL-17F and M-CSF; concentration-dependent effects observed with sIL-17F within HDFSAg; Tissue remodeling activities: decreased EGFR and sTGF-b; increased Col-I; concentration-dependent effects observed with sVEGF in HDFSAg and HDFLPS.

### Aspartame

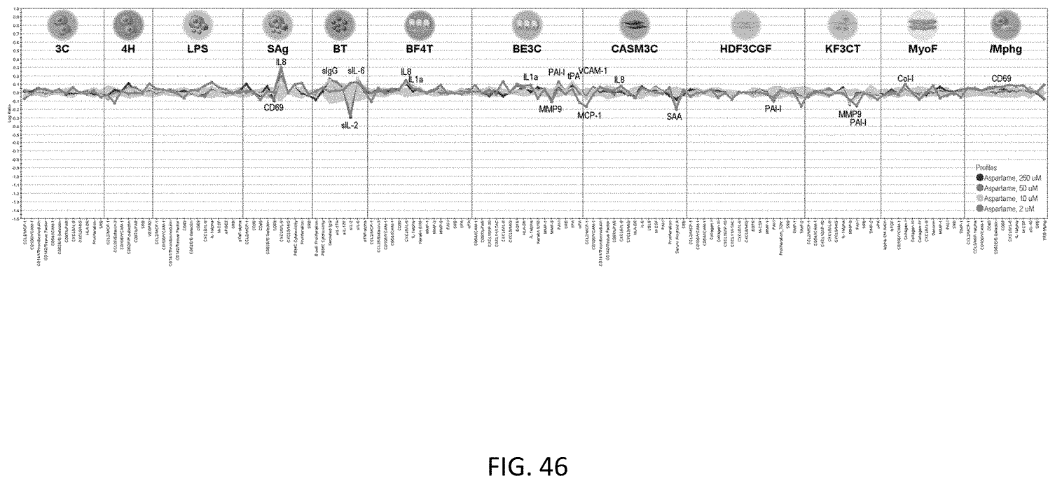

FIG. 46 provides a first activity profile of aspartame. The key activities of aspartame include inflammation-related activities: decreased MCP-1 and SAA; increased IL-8, IL-1α and VCAM-1; immunomodulatory activities: decreased sIL-2; increased sIgG and sIL-6; modulated CD69; and tissue remodeling activities: decreased MMP-9; increased Collagen-I and tPA; modulated PAI-I.

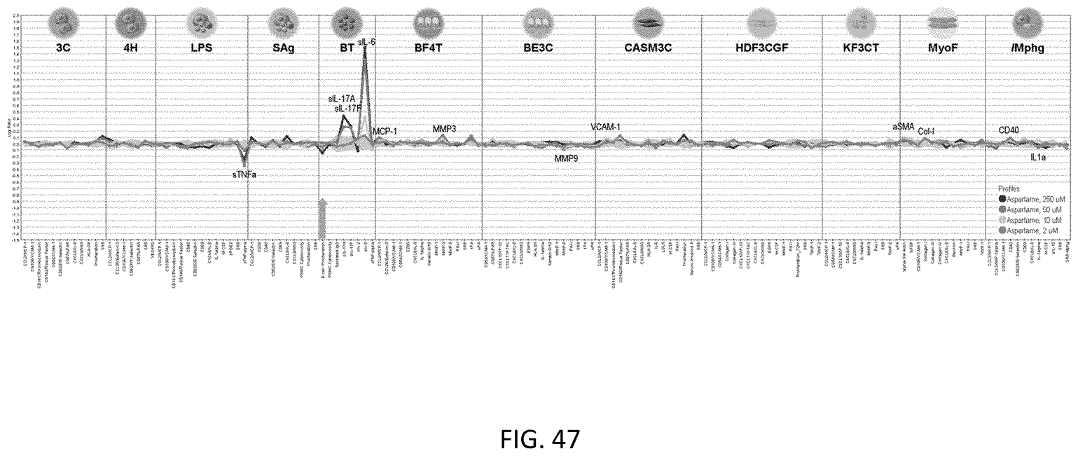

FIG. 47 provides a second activity profile of aspartame. The key activities of aspartame include anti-proliferative to B cells (grey arrow); inflammation-related activities: decreased sTNFα and IL1α; increased MCP-1 and VCAM-1; immunomodulatory activities: increased sIL-17A, sIL-17F, sIL-6, and CD40; and tissue remodeling activities: decreased MMP9; increased MMP3, αSMA, and Col-I.

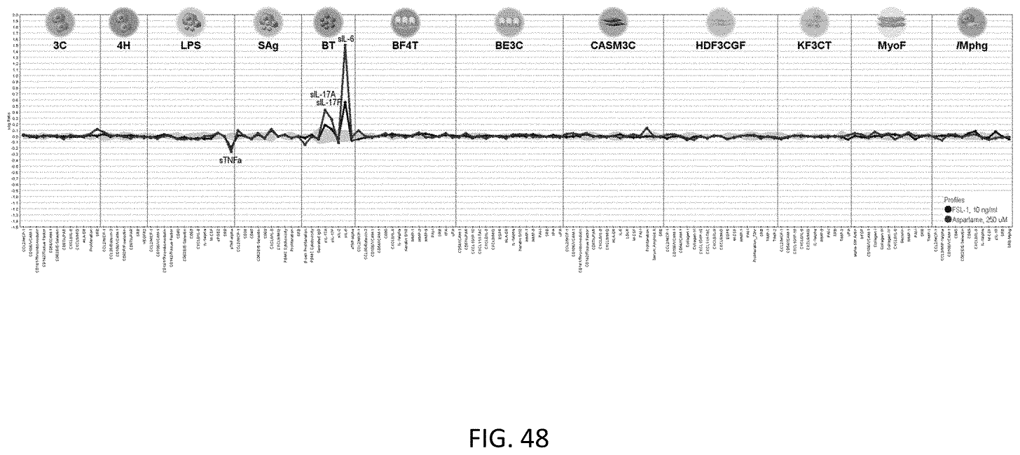

A comparison of aspartame and FSL-1 is provided in FIG. 48. The overlay of aspartame (250 μM) and FSL-1 (10 ng/ml) has a Pearson's correlation, r=0.909; the common activities are annotated. FSl-1 is a bacterial-derived toll-like receptor 2/6 agonist.

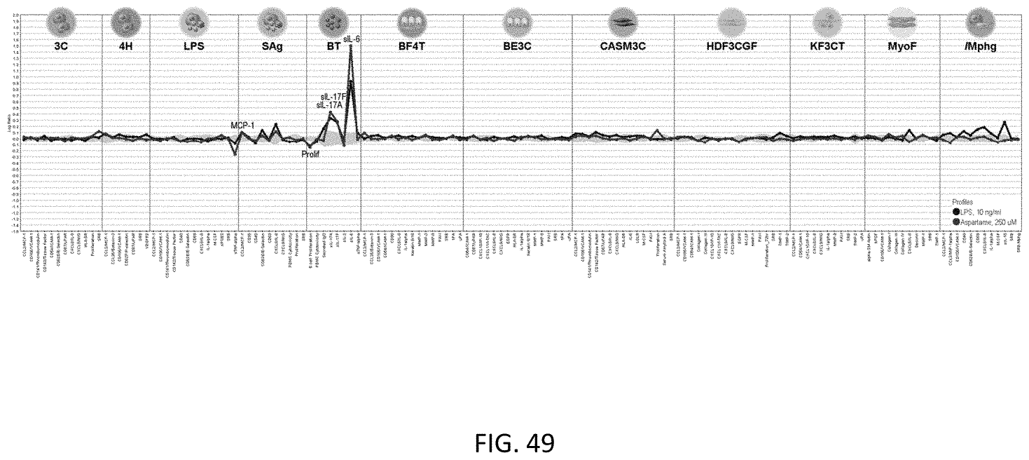

A comparison of aspartame and LPS is provided in FIG. 49. The overlay of aspartame has a Pearson's correlation, r=0.879; common activities are annotated. LPS, also known as lipoglycans and endotoxin, are large molecules consisting of lipid and polysaccharide found in the outer membrane of Gram-negative bacteria.

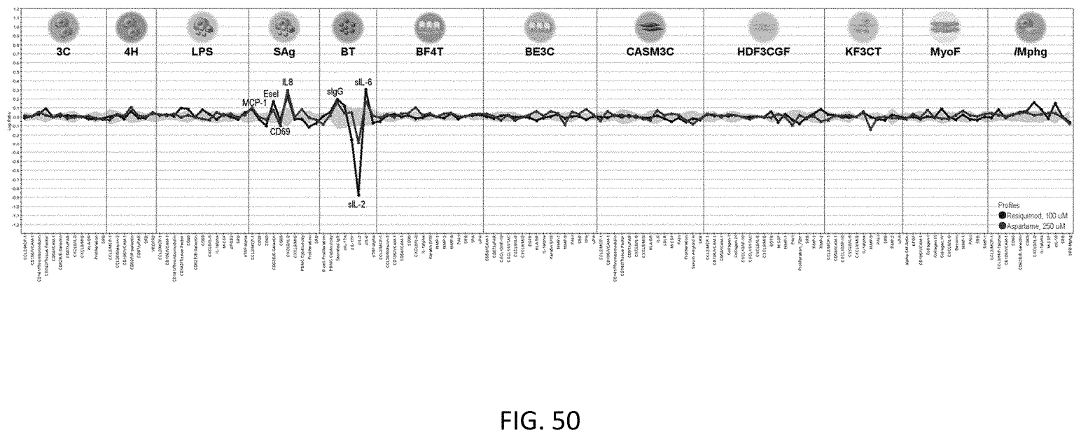

A comparison of aspartame (250 μM) and resiquimod (100 μM) is provided in FIG. 50. The overlay of aspartame has a Pearson correlation, r=0.626; the relevance of similarities at Pearson's correlations, r<0.7 is unknown; common activities are annotated. Resiquimod is a potent synthetic agonist of TLR7/TLR8 that possesses anti-viral and anti-tumoral activities. Resiquimod is used topically for the treatment of viral skin lesions as well as an adjuvant to increase the effectiveness of vaccines.

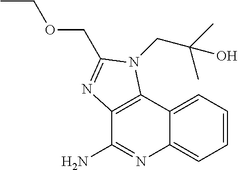 [SMILES: CCOCc1nc2c(N)nc3ccccc3c2n1CC(C)(C)O]

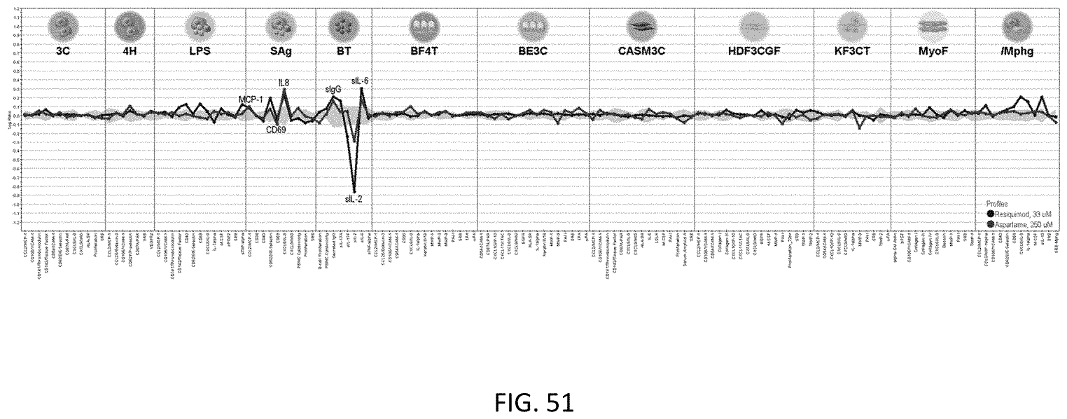

A comparison of aspartame (250 μM) and resiquimod (33 μM) is provided in FIG. 51. The overlay of aspartame has a Pearson correlation, r=0.608; the relevance of similarities at Pearson's correlations, r<0.7 is unknown; common activities are annotated.

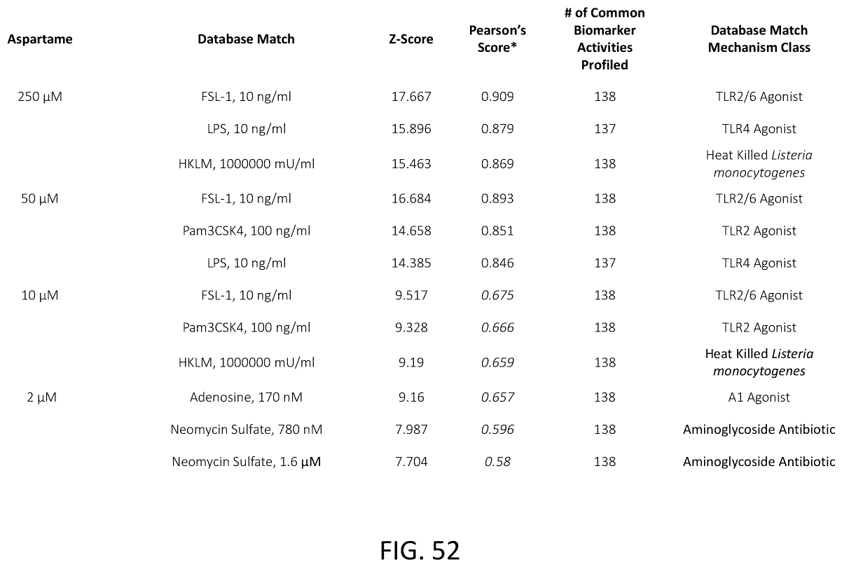

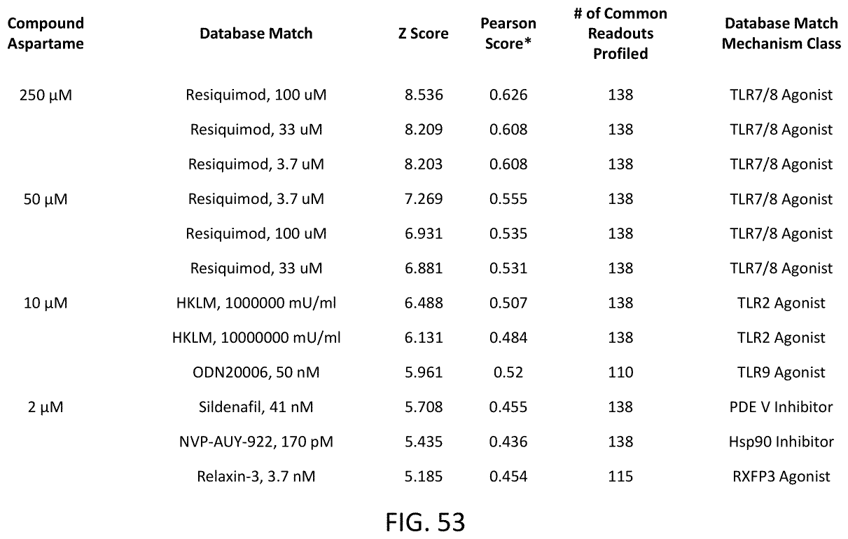

Top reference database matches for aspartame and various compounds, along with Z-score, Pearson's Score, number of common biomarker activities profiled, and database match to mechanism class are provided in FIG. 52 and FIG. 53. Pearson's correlations with r<0.7 indicate the relevance of similarity is unknown.

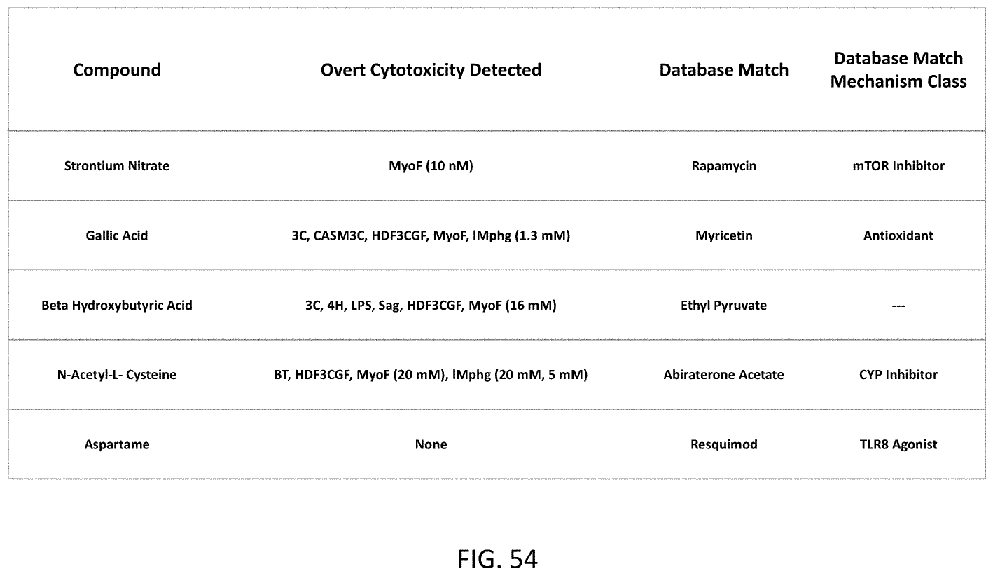

FIG. 54 is a summary table of data providing overt cytotoxicity data, database match, and mechanism class information for various compounds. The data indicate that strontium nitrate has activity as a mTOR inhibitor, gallic acid has activity as an antioxidant, N-acetyl-L-cysteine has activity as a CYP inhibitor, and aspartame has activity as a TLR8 agonist.

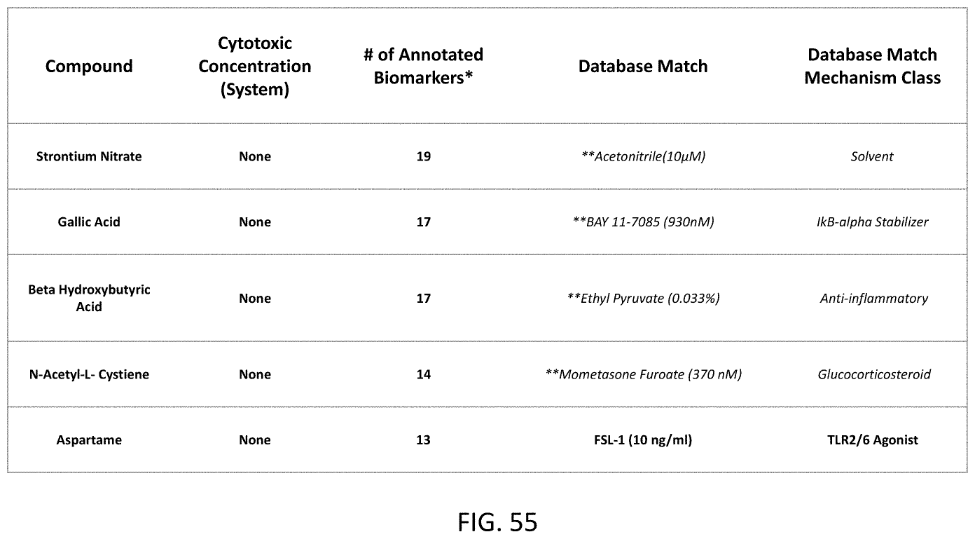

FIG. 55 is a summary table of data providing cytotoxic concentration (system, if any), number of annotated biomarkers (if the endpoint value at 2 or more consecutive concentrations is outside the 95% vehicle control envelope in the same direction), database match (** database similarity match is below the Pearson's correlation threshold of 0.7; relevance of match is unknown), and mechanism class. The data indicate that strontium nitrate has solvent activity, gallic acid is an IkB-alpha stabilizer, beta hydroxybutyric acid exhibits antiinflammatory activity, N-acetyl-L-cystiene is a glucocorticosteroid, and aspartame is a TLR2/6 agonist.

### Exemplary Methods

Method 1: A method for modulating production of a cytokine, or molecule upregulated or down regulated by a cytokine, comprising: administering to a patient in need thereof an effective amount of a composition comprising at least one compound selected from the group consisting of a strontium-containing compound, a cysteine-based antioxidant, a polyhydroxyphenol and beta hydroxybutyric acid, whereby an amount of a cytokine in the patient is modulated.

Method 2: Method 1, wherein the composition comprises a complex of a strontium-containing compound, a cysteine-based antioxidant, and at least one of polyhydroxyphenol and beta hydroxybutyric acid, wherein the cysteine-based anti-oxidant and the at least one of polyhydroxyphenol and beta hydroxybutyric acid are optionally conjugated together by a cleavable bond.

Method 3: Method 1, wherein the cytokine is associated with inflammation.

Method 4: Method 3, wherein the cytokine or a molecule upregulated or down regulated by the cytokine is selected from the group consisting of Eotaxin-3, soluble tumor necrosis factor-alpha (sTNF-α), vascular cell adhesion molecule 1 (VCAM-1), interleukin-1 alpha (IL-1α), interferon gamma-induced protein 10 (IP-10), interferon-inducible T-cell alpha chemoattractant (I-TAC), interleukin-8 (IL-8), serum amyloid A protein (SAA), monocyte chemo attractant protein-1 (MCP-1), prostaglandin E2 (sPGE2), intercellular adhesion molecule 1 (ICAM-1), monokine induced by interferon-gamma (MIG) (need to identify this molecule), E-selectin, P-selectin, macrophage inflammatory protein 1 (MIP-1) and interleukin 6 (IL-6).

Method 5: Method 3, wherein the cytokine or a molecule upregulated or down regulated by the cytokine is associated with a disorder selected from the group consisting of acne, psoriasis, rosacea, atopic dermatitis and eczema.

Method 6: Method 1, wherein the cytokine or a molecule upregulated or down regulated by said cytokine is associated with immunomodulation.

Method 7: Method 6, wherein the cytokine or a molecule upregulated or down regulated by the cytokine is selected from the group consisting of human leukocyte antigen-antigen D related (HLA-DR), soluble immunoglobulin G (sIgG), soluble interleukin 17A (sIL-17A), [symbol] ), soluble interleukin 17F (sIL-17F), soluble interleukin 6 (sIL-6), macrophage colony-stimulating factor (M-CSF), soluble interleukin 2 (sIL-2), soluble interleukin 10 (sIL-10), cluster of differentiation 40 (CD40) and cluster of differentiation 69 (CD69).

Method 8: Method 1, wherein the cytokine or a molecule upregulated or down regulated by the cytokine is associated with tissue remodeling.

Method 9: Method 8, wherein the cytokine or a molecule upregulated or down regulated by the cytokine is selected from the group consisting of urokinase-type plasminogen activator (uPAR or CD87), matrix metalloproteinase 1 (MMP-1), matrix metalloproteinase 9 (MMP-9), plasminogen activator inhibitor 1 (PAI-I), tissue plasminogen activator (tPA), matrix metalloproteinase 3 (MMP-3), Keratin 8/18, TIMP metallopeptidase inhibitor 2 (TIMP-2), basic fibroblast growth factor (bFGF), Collagen-III, alpha smooth muscle actin antibody (α-SMA), Collagen-IV, epidermal growth factor receptor (EGFR), Collagen-I and TIMP metallopeptidase inhibitor 1 (TIMP-1).

Method 10: Method 8, wherein the tissue remodeling is associated with a tissue injury or a tissue trauma.

Method 11: Method 1, wherein the strontium-containing compound is selected from the group consisting of strontium nitrate, strontium chloride and strontium chloride hexahydrate.

Method 12: Method 1, wherein the polyhydroxyphenol is gallic acid.

Method 13: Method 1, wherein the cysteine-based antioxidant is selected from the group consisting of N-acetyl-cysteine, cysteine, cystine, acetylcysteine, diacetylcysteine, and esters thereof.

Method 14: Method 1, wherein the polyhydroxyphenol is selected from the group consisting of gallic acid, caffeic acid, tannic acid, epicatechin, epigallocatechin gallate, epigallocatechin, epicatechin gallate, ellagic acid, myricetin, luteolin, naringen, genistein, apagenin, nordihydroguaiaretic acid, and esters thereof.

Method 15: Method 1, wherein production of the cytokine or a molecule upregulated or down regulated by the cytokine is increased.

Method 16: Method 1, wherein production of a molecule is upregulated or down regulated by the cytokine.

Method 17: Method 1, wherein the composition is topically administered, optionally in a formulation selected from the group consisting of a gel, emulsion, lotion, creams, ointment, pastes, and salve.

Method 18: Method 1, wherein the composition is orally administered, optionally in a formulation selected from the group consisting of a tablet, pill, dragee, capsule, liquid, gel, syrup, elixir, slurry and suspension.

Method 19: Method 1, wherein the composition consists of a strontium-containing compound, a cysteine-based antioxidant and a polyhydroxyphenol, wherein the strontium-containing compound is strontium chloride, the cysteine-based antioxidant is N-acetyl cysteine, and the polyhydroxyphenol is gallic acid.

Method 20: Method 1, wherein the composition consists of a strontium-containing compound, a cysteine-based antioxidant and beta hydroxybutyrate, wherein the strontium-containing compound is strontium hydroxide and the cysteine-based antioxidant is N-acetyl cysteine.

What is claimed is:

1. A method for modulating production of a cytokine, or molecule upregulated or down regulated by a cytokine, comprising:
administering to a patient in need thereof an effective amount of a composition comprising at least one compound selected from the group consisting of a strontium-containing compound, a cysteine-based antioxidant, a polyhydroxyphenol and beta hydroxybutyric acid, whereby an amount of a cytokine in the patient is modulated.

2. The method of claim 1, wherein the composition comprises a complex of a strontium-containing compound, a cysteine-based antioxidant, and at least one of polyhydroxyphenol and beta hydroxybutyric acid, wherein the cysteine-based anti-oxidant and the at least one of polyhydroxyphenol and beta hydroxybutyric acid are optionally conjugated together by a cleavable bond.

3. The method of claim 1, wherein the cytokine is associated with inflammation.

4. The method of claim 3, wherein the cytokine or a molecule upregulated or down regulated by the cytokine is selected from the group consisting of Eotaxin-3, soluble tumor necrosis factor-alpha (sTNF-α), vascular cell adhesion molecule 1 (VCAM-1), interleukin-1 alpha (IL-1α), interferon gamma-induced protein 10 (IP-10), interferon-inducible T-cell alpha chemoattractant (I-TAC), interleukin-8 (IL-8), serum amyloid A protein (SAA), monocyte chemoattractant protein-1 (MCP-1), prostaglandin E2 (sPGE2), intercellular adhesion molecule 1 (ICAM-1), monokine induced by interferon-gamma (MIG) (need to identify this molecule), E-selectin, P-selectin, macrophage inflammatory protein 1 (MIP-1) and interleukin 6 (IL-6).

5. The method of claim 3, wherein the cytokine or a molecule upregulated or down regulated by the cytokine is associated with a disorder selected from the group consisting of acne, psoriasis, rosacea, atopic dermatitis and eczema.

6. The method of claim 1, wherein the cytokine or a molecule upregulated or down regulated by said cytokine is associated with immunomodulation.

7. The method of claim 6, wherein the cytokine or a molecule upregulated or down regulated by the cytokine is selected from the group consisting of human leukocyte antigen-antigen D related (HLA-DR), soluble immunoglobulin G (sIgG), soluble interleukin 17A (sIL-17A), [symbol] ), soluble interleukin 17F (sIL-17F), [symbol] ), soluble interleukin 6 (sIL-6), macrophage colony-stimulating factor (M-CSF), soluble interleukin 2 (sIL-2), soluble interleukin 10 (sIL-10), cluster of differentiation 40 (CD40) and cluster of differentiation 69 (CD69).

8. The method of claim 1, wherein the cytokine or a molecule upregulated or down regulated by the cytokine is associated with tissue remodeling.

9. The method of claim 8, wherein the cytokine or a molecule upregulated or down regulated by the cytokine is selected from the group consisting of urokinase-type plasminogen activator (uPAR or CD87), matrix metalloproteinase 1 (MMP-1), matrix metalloproteinase 9 (MMP-9), plasminogen activator inhibitor 1 (PAI-I), tissue plasminogen activator (tPA), matrix metalloproteinase 3 (MMP-3), Keratin 8/18, TIMP metallopeptidase inhibitor 2 (TIMP-2), basic fibroblast growth factor (bFGF), Collagen-III, alpha smooth muscle actin antibody (α-SMA), Collagen-IV, epidermal growth factor receptor (EGFR), Collagen-I and TIMP metallopeptidase inhibitor 1 (TIMP-1).

10. The method of claim 8, wherein the tissue remodeling is associated with a tissue injury or a tissue trauma.

11. The method of claim 1, wherein the strontium-containing compound is selected from the group consisting of strontium nitrate, strontium chloride and strontium chloride hexahydrate.

12. The method of claim 1, wherein the polyhydroxyphenol is gallic acid.

13. The method of claim 1, wherein the cysteine-based antioxidant is selected from the group consisting of N-acetyl-cysteine, cysteine, cystine, acetylcysteine, diacetylcysteine, and esters thereof.

14. The method of claim 1, wherein the polyhydroxyphenol is selected from the group consisting of gallic acid, caffeic acid, tannic acid, epicatechin, epigallocatechin gallate, epigallocatechin, epicatechin gallate, ellagic acid, myricetin, luteolin, naringen, genistein, apagenin, nordihydroguaiaretic acid, and esters thereof.

15. The method of claim 1, wherein production of the cytokine or a molecule upregulated or down regulated by the cytokine is increased.

16. The method of claim 1, wherein production of a molecule is upregulated or down regulated by the cytokine.

17. The method of claim 1, wherein the composition is topically administered, optionally in a formulation selected from the group consisting of a gel, emulsion, lotion, creams, ointment, pastes, and salve.

18. The method of claim 1, wherein the composition is orally administered, optionally in a formulation selected from the group consisting of a tablet, pill, dragee, capsule, liquid, gel, syrup, elixir, slurry and suspension.

19. The method of claim 1, wherein the composition consists of a strontium-containing compound, a cysteine-based antioxidant and a polyhydroxyphenol, wherein the strontium-containing compound is strontium chloride, the cysteine-based antioxidant is N-acetyl cysteine, and the polyhydroxyphenol is gallic acid.

20. The method of claim 1, wherein the composition consists of a strontium-containing compound, a cysteine-based antioxidant and beta hydroxybutyrate, wherein the strontium-containing compound is strontium hydroxide and the cysteine-based antioxidant is N-acetyl cysteine.
